(19) United States
(12) Patent Application Publication
(10) Pub. No.: US 2006/0014813 A1
(43) Pub. Date: Jan. 19, 2006

(54) Pharmaceutical compounds that regenerate in vivo

(76) Inventors: Patrick Connelly, Harvard, MA (US); Gregory Connelly, Vienna (AT); Andrew Magee, Maynard, MA (US)

Correspondence Address: VERTEX PHARMACEUTICALS INC., 130 WAVERLY STREET, CAMBRIDGE, MA, 02139-4242, US

(21) Appl. No.: 10/997,752

(22) Filed: Nov. 24, 2004

Related U.S. Application Data
(60) Provisional application No. 60/525,209, filed on Nov. 26, 2003.

(51) Int. Cl. A61K 31/42; A61K 31/415; C07D 231/10; C07D 261/06

(52) U.S. Cl. 514/378; 514/406; 548/247; 548/377.1

(57) ABSTRACT

The invention relates to a class of compounds that reacts with and neutralizes a reactive oxygen species, such as a free oxygen radical, in a patient and which can then be regenerated back to their original reactive chemical form by a naturally occurring enzyme in said patient. These compounds are useful to treat diseases in a patient characterized by a reactive oxygen species. Moreover, because these compounds can be regenerated back to their original, reactive chemical state in vivo, a single molecule can neutralize multiple molecules of the reactive species. This allows for the use of lower dosages for the treatment of disease, as compared to compounds presently used to treat that same disease, thus avoiding side effects associated with higher dosages.

### TECHNICAL FIELD OF THE INVENTION

The invention relates to a class of compounds that reacts with and neutralizes a reactive oxygen species, such as a free oxygen radical, in a patient and which can then be regenerated back to their original reactive chemical form by a naturally occurring enzyme in said patient. These compounds are useful to treat diseases in a patient characterized by a reactive oxygen species. Moreover, because these compounds can be regenerated back to their original, reactive chemical state in vivo, a single molecule can neutralize multiple molecules of the reactive species. This allows for the use of lower dosages for the treatment of disease, as compared to compounds presently used to treat that same disease, thus avoiding side effects associated with higher dosages.

### BACKGROUND OF THE INVENTION

Reactive oxygen species (“ROS”), such as O₂—, OH radicals and H₂O₂, form in cells during the metabolism of oxygen, especially in the reduction of oxygen by the electron transfer system of mitochondria. These three species are generated in cells through the action of enzymes, such as cytochrome c oxidase, NADPH-oxidase and flavin enzymes, as well as radiation (e.g., UV light), toxic chemicals (e.g., paraquat) and drugs (e.g., adriamycin, bleomycin).

ROS can react with all biological macromolecules (lipids, proteins, nucleic acids and carbohydrates) causing significant damage. For example, oxidation of proteins can cause modification of both the structure and function. Metal-catalyzed protein oxidation results in addition of carbonyl groups or cross-linking or fragmentation of proteins. Aldehydes resulting from peroxidation of lipids can react with cysteine or basic amino acids (histidine, lysine). Similarly, modification of individual nucleotide bases, singlestrand breaks and cross-linking are the typical effects of reactive oxygen species on nucleic acids.

These reactions, termed “oxidative stress,” have been linked to many diseases and conditions. These include arterial disease, heart and pulmonary diseases, rheumatoid disease, eye disease (cataract, macular degeneration), gum disease, respiratory disease, Sickle Cell Anemia, ischemia/reperfusion injuries, neurodegenerative diseases (ALS, Alzheimer's Disease, Huntington's disease, Parkinson's disease), inflammation (chronic and acute), cancer, reproductive dysfunction and aging. Indeed, CoQ (an antioxidant dietary supplement) is currently being investigated clinically for treating patients with Parkinson's disease.

Methionine sulfoxide reductases (MSR; msR; msr) are a class of enzymes that process harmful oxidation by-products and repair oxidatively damaged proteins. Oxidation of methionine by ROS yields methionine sulfoxide, which is converted back to methionine by MSR. The role of MSRs in protecting against oxidative damage that contributes to cell death has been biologically validated in a variety of living systems, in vivo as well as in vitro. Unpublished research has recently shown neuroprotective effects for MSRs in a validated animal model of a neurodegenerative disease. It has also shown life-extension in an animal model.

Over-expression of MSRs extends life and protects against oxidative damage. Knocking out MSR genes produces neurological lesions in mice and Drosophila, illustrating their critical importance in the nervous system, and causes those species to die younger than control organisms. Also, Drosophila that overexpress MSR demonstrate an extended lifespan. MSRs have also been shown to counteract disease states in cellular systems.

MSR, however, being a large enzyme is not readily suitable as a therapeutic. Thus, there is a need for a small molecule therapeutic that can somehow utilize the in vivo activity of MSR to reduce or eliminate the amount of ROS in a cell. Such a compound that can reduce or eliminate the accumulation of excessive oxidation byproducts of the cell that lead to or exacerbate various disease states is highly desirable. Moreover, a compound that both possessed antioxidant activity and was able to neutralize greater than a single mole of ROS per mole of compound would be even more preferred. Such a compound could be administered in lower doses and thus avoid unwanted side effects typical of drugs when administered in higher dosages.

Neurodegeneration due to ROS has been a longstanding and leading pathogenic hypothesis [Beal (2002) Ann. Neurol. 53 (S3): 39-48]. It has been shown that blocking COX-2 activity leads to neuroprotection in animals. Similarly, knocking out NADPH-oxidase in mice leads to diminished neuronal loss in an animal model of Parkinson's Disease [Moscovitz et al. (1995) J. Bacteriology 177: 502-507; Ruan et al. (2002) Proc. Nat. Acad. Sci. USA 99:2748-2753.]

COX-2 inhibitors are described in, for example, U.S. Pat. Nos. 5,563,1654, 5,691,374, 5,760,068, 5,972,986, 5,644,272, and 6,239,173. It has been suggested that COX-2 inhibitors may be useful as neuroprotective agents. Indeed, Celebrex and Vioxx both provide a partial recovery of dopamine production (˜20%) in animal models of Parkinson's disease. Similar partial therapeutic benefits have been observed in animal models of ALS. Celebrex is being tested in a large phase II/III clinical trial for ALS and it is expected that trials of Celebrex and Vioxx for Parkinson's disease will begin shortly.

There is an older report in the literature that suggests that difusinal [5-(2′-4′-difluorophenyl) salicylic acid] possesses both ant-inflammatory and oxygen radical scavenging activities [T. Y. Shen, Pharmacotherapy, 2, pp. 3S-8S (1983)]. However, both activities appear to be due to the same moiety and the anti-oxidant activity is not regenerable. Furthermore, the anti-inflammatory activity does not specifically target COX-2 without also inhibiting COX-1 since this compound is a derivative of aspirin.

A compound which could combine COX-2 inhibitory activity and antioxidant activity would therefore be extremely useful in the treatment of neurodegenerative diseases, as well as any other disease that involved inflammation and oxidative damage.

### SUMMARY OF THE INVENTION

Applicant has solved the problem set forth above by providing compounds which serve as a “sink” for ROS and which can then be regenerated back to an active form through the action of MSR. One mole of the compounds of the present invention can inactivate many moles of ROS. Most drugs act stoichiometrically; that is, each unit of drug administered gives rise to one unit of therapeutic benefit. Each unit of a compound of the present invention administered gives rise to many units of therapeutic benefit. The compounds of this invention are designed to leverage the body's own MSR enzymes to continually regenerate the administered drug after it has produced its therapeutic effect.

The compounds of the present invention lead to a reduction of ROS in the cell and can serve as a treatment for or prevention of diseases and conditions associated with ROS. Such diseases include arterial disease, heart and pulmonary diseases, rheumatoid disease, eye disease (cataract, macular degeneration), gum disease, respiratory disease, Sickle Cell Anemia, ischemia/reperfusion injuries, neurodegenerative diseases (ALS, Alzheimer's disease, Huntington's disease, Parkinson's disease), inflammation (chronic and acute), cancer, and reproductive dysfunction.

The compounds of the present invention are useful for the treatment of diseases, disorders, and conditions including, but not limited to acute, chronic, neuropathic, or inflammatory pain, arthritis, migrane, cluster headaches, trigeminal neuralgia, herpetic neuralgia, general neuralgias, epilepsy or epilepsy conditions, neurodegenerative disorders, psychiatric disorders such as anxiety and depression, myotonia, arrythmia, movement disorders, neuroendocrine disorders, ataxia, multiple sclerosis, irritable bowel syndrome, and incontinence. Accordingly, in another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

According to one embodiment, the compounds of the present invention are useful for treating a disease selected from femur cancer pain; non-malignant chronic bone pain; rheumatoid arthritis; osteoarthritis; spinal stenosis; neuropathic low back pain; neuropathic low back pain; myofascial pain syndrome; fibromyalgia; temporomandibular joint pain; chronic visceral pain, including, abdominal; pancreatic; IBS pain; chronic headache pain; migraine; tension headache, including, cluster headaches; chronic neuropathic pain, including, post-herpetic neuralgia; diabetic neuropathy; HIV-associated neuropathy; trigeminal neuralgia; Charcot-Marie Tooth neuropathy; hereditary sensory neuropathies; peripheral nerve injury; painful neuromas; ectopic proximal and distal discharges; radiculopathy; chemotherapy induced neuropathic pain; radiotherapy-induced neuropathic pain; post-mastectomy pain; central pain; spinal cord injury pain; post-stroke pain; thalamic pain; complex regional pain syndrome; phanton pain; intractable pain; acute pain, acute post-operative pain; acute musculoskeletal pain; joint pain; mechanical low back pain; neck pain; tendonitis; injury/exercise pain; acute visceral pain, including, abdominal pain; pyelonephritis; appendicitis; cholecystitis; intestinal obstruction; hernias; etc; chest pain, including, cardiac Pain; pelvic pain, renal colic pain, acute obstetric pain, including, labor pain; cesarean section pain; acute inflammatory, burn and trauma pain; acute intermittent pain, including, endometriosis; acute herpes zoster pain; sickle cell anemia; acute pancreatitis; breakthrough pain; orofacial pain, including, sinusitis pain, dental pain; multiple sclerosis (MS) pain; pain in depression; leprosy pain; behcet's disease pain; adiposis dolorosa; phlebitic pain; Guillain-Barre pain; painful legs and moving toes; Haglund syndrome; erythromelalgia pain; Fabry's disease pain; bladder and urogenital disease, including, urinary incontinence; hyperactivity bladder; painful bladder syndrome; interstitial cyctitis (IC); and prostatitis.

The compounds of the invention have the chemical formula: 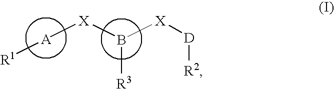
wherein:

A is selected from aryl or heteroaryl;

ring B is selected from heterocyclyl, or carbocyclyl and is optionally benzofused;

D is selected from heterocyclyl, carbocyclyl, heteroaryl, or aryl and, when B is benzofused, is additionally selected from —(C₁-C₆)-alkylidene- or —(C₁-C₆)-heteroalkylidene-;

wherein each of A, B and D is optionally additionally substituted with one or more independently selected substitutents;

each X is independently selected from a bond, —C(O)—, —CH₂—, —CH═, or ═CH—;

each or R¹, R² and R³ is independently selected from hydrogen, methyl, or -L-SO)ₘR⁴; R³ is additionally selected from CF₃ or C(O)OR^{o} when B comprises only one substitutable ring atom; and at least one of R¹, R² and R³ is selected from -L-S—CH₃ optionally substituted with halo, O-methyl, O-ethyl, or CN; -L-S—CH₂CH₃ optionally substituted with halo, O-methyl, or CN; -L-S—CH₂CH₂CH₃ optionally substituted with halo; or -L-S—CH(CH₃)CH₃ optionally substituted with halo, wherein:

each m is independently 0, 1 or 2;

each L is independently selected from a bond, an optionally substituted (C₁-C₆)-alkylidene or, an optionally substituted (C₁-C₆)-heteroalkylidene;

R^{o} is selected from hydrogen, optionally substituted C₁₋₆ aliphatic, an unsubstituted carbocyclic ring, an unsubstituted 5-6 membered heteroaryl or heterocyclic ring, an unsubstituted aryl ring, —O-aryl, or —CH₂-aryl, wherein said substituents on the aliphatic group of R^{o} are independently selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of R^{o} is unsubstituted; and

each R⁴ is independently selected from hydrogen, unsubstituted aryl, unsubstituted heteroaryl, unsubstituted carbocyclyl, unsubstituted heterocyclyl, —NH—(C₁-C₃)-aliphatic, —O—(C₁-C₃)-aliphatic or —(C₁-C₆)-aliphatic;

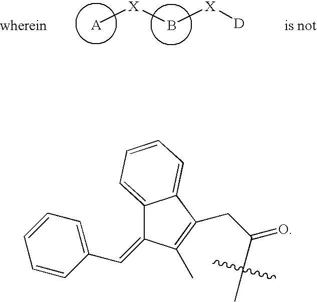

Certain of the compounds of the present invention also possess, in addition to an anti-oxidant activity, a COX-2 inhibitory activity. Such dual-action compounds are especially useful in the treatment of diseases and conditions characterized by inflammation and oxidative stress. These diseases include neurodegenerative disease, such as Parkinson's disease, Alzheimer's disease and ALS, inflammation, cardiovascular diseases, reperfusion injuries, diseases of the eye, and cancer.

The compounds of this invention may be formulated into compositions for administration to animals and the resulting composition used in methods for the prevention or treatment of any of the diseases set forth above.

### BRIEF DESCRIPTION OF THE DRAWINGS

FIG. 1 depicts the results of a calorimetric assay to determine if methionine sulfoxide or methionine sulfone is turned over by msrA. Three separate injections of compound (arrows) were made into a calorimetric cell containing msrA and the heat generated following injection is measured.

FIG. 2 depicts the results of a calorimetric assay to determine if compound 100 is a substrate for msrA. Multiple injections of compound 100 were made into a calorimetric cell containing msrA and the heat generated following each injection is measured.

FIG. 3 depicts a plot of the rate of reaction between various concentrations of compound 100 and msrA derived from the data in FIG. 2. The inset to FIG. 3 depicts the value or various parameters derived from the plot.

FIG. 4 shows a representation of cyclooxygenase-2 bound to indomethacin.

### DETAILED DESCRIPTION OF THE INVENTION

As used herein, the following definitions shall apply unless otherwise indicated. The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring.”

The term “heteroaryl”, used alone or as part of a larger moiety, as in “heteroaralkyl” or “heteroarylalkoxy,” refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR₊(as in N-substituted pyrrolidinyl)).

The term or “carbocycle,” “carbocyclic,” or “carbocyclyl” refers to a monocyclic C₃-C₈ hydrocarbon or bicyclic C₈-C₁₂ hydrocarbon that is completely saturated or that contains one or more units of unsaturation, but which is not aromatic and that has a single point of attachment to the rest of the molecule.

The term “heterocycle,” “heterocyclyl,” or “heterocyclic” as used herein means non-aromatic, monocyclic, bicyclic, or tricyclic ring systems having three to fourteen ring members in which one or more ring members is a heteroatom independently selected from oxygen, sulfur, nitrogen, or phosphorus, and each ring in the system contains 3 to 7 ring members.

The phrase “optionally substituted” is used interchangeably with the phrase “substituted or unsubstituted.” The term “substituted” means the replacement of a hydrogen atom with another moiety. For the purpose of clarity, substitutions may also occur at terminal hydrogen atoms. Unless otherwise indicated, an optionally substituted group may have a substituent at each substitutable position of the group, and each substitution is independent of the other. Combinations of substituents envisioned by this invention are preferably those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Suitable substituents on an unsaturated carbon atom of an aryl (including the phenyl group that is optionally benzofused to B) or heteroaryl group are selected from halogen; —R^{o}; —OR^{o}; —SR^{o}; 1,2-methylenedioxy; 1,2-ethylenedioxy; phenyl (Ph) optionally substituted with R^{o}; —O(Ph) optionally substituted with R^{o}; —(CH₂)₁₋₂(Ph), optionally substituted with R^{o}; —CH═CH(Ph), optionally substituted with R^{o}; —NO₂; —CN; —N(R^{o})₂; —NR^{o}C(O)R^{o}; —NR^{o}C(O)N(R^{o})₂; —NR^{o}CO₂R^{o}; —NR^{o}NR^{o}C(O)R^{o}; —NR^{o}NR^{o}C(O)N(R^{o})₂; —NR^{o}NR^{o}CO₂R^{o}; —C(O)C(O)R^{o}; —C(O)CH₂C(O)R^{o}; —CO₂R^{o}; —C(O)R^{o}; —C(O)N(R^{o})₂; —OC(O)N(R^{o})₂; —S(O)₂R^{o}; —SO₂N(R^{o})₂; —S(O)R^{o}; —NR^{o}SO₂N(R^{o})₂; —NR^{o}SO₂R^{o}; —C(═S)N(R^{o})₂; —C(═NH)—N(R^{o})₂; or —(CH₂)₀₋₂NHC(O)R^{o}, wherein each independent occurrence of R^{o} is selected from hydrogen, optionally substituted C₁₋₆ aliphatic, an unsubstituted carbocyclic ring, an unsubstituted 5-6 membered heteroaryl or heterocyclic ring, an unsubstituted aryl ring, —O-aryl, or —CH₂-aryl, or, notwithstanding the definition above, two independent occurrences of R^{o}, on the same substituent or different substituents, taken together with the atom(s) to which each R^{o} group is bound, form a 3-8-membered cycloalkyl, heterocyclyl, aryl, or heteroaryl ring having 0-3 heteroatoms independently selected from nitrogen, oxygen, or sulfur. Optional substituents on the aliphatic group of R^{o} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of R^{o} is unsubstituted.

The term “aliphatic” or “aliphatic group” as used herein means a straight chain orbranched C₁-C₁₂ hydrocarbon chain that is completely saturated or that contains one or more units of unsaturation. Preferably, an aliphatic group is a straight chain or branched C₁-C₆ hydrocarbon chain. For example, suitable aliphatic groups include, but are not limited to, linear or branched or alkyl, alkenyl, alkynyl groups. Aliphatic groups may be substituted or unsubstituted, unless otherwise indicated.

The term “heteroaliphatic”, as used herein, means aliphatic groups wherein one or two carbon atoms are independently replaced by one or more of oxygen, sulfur, nitrogen, phosphorus, or silicon. Heteroaliphatic groups of specific lengths are indicated as “Cₙ-heteroaliphatic,” wherein n is the number of carbon atoms in the heteroaliphatic prior to the replacement of one or two with oxygen, sulfur, nitrogen, phosphorus, or silicon. Heteroaliphatic groups may be substituted or unsubstituted, branched or unbranched unless otherwise indicated.

The term “alkylidene” refers to a straight or branched C₁-C₁₂ hydrocarbon chain that may be fully saturated or have one or more units of unsaturation and has two points of attachment to the rest of the molecule.

The term “heteroalkylidene” refers to an alkylidene wherein one or two carbon atoms are independently replaced by one or more of oxygen, sulfur, nitrogen, phosphorus, or silicon. Heteroaliphatic groups of specific lengths are indicated as “Cₙ-heteroalkylidene,” wherein n is the number of carbon atoms in the heteroalkylidene prior to the replacement of one or two with oxygen, sulfur, nitrogen, phosphorus, or silicon. Heteroalkylidene groups may be substituted or unsubstituted, branched or unbranched unless otherwise indicated.

The terms “alkyl”, “alkenyl” and “alkynyl” used alone or as part of a larger moiety shall include both straight and branched chains.

The terms “haloalkyl,” “haloalkenyl,” and “haloaliphatic” means alkyl, alkenyl or aliphatic, as the case may be, substituted with one or more halogen atoms. The terms “halogen” and “halo” means F, Cl, Br, or I.

An alkylidene, heteroalkylidene, aliphatic or heteroaliphatic group, or a nonaromatic carbocyclic or heterocyclic ring may contain one or more substituents. Suitable substituents on the saturated carbon of an aliphatic or heteroaliphatic group, or of a nonaromatic heterocyclic ring are selected from those listed above for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, where each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R* is unsubstituted.

Optional substituents on the nitrogen of a non-aromatic heterocyclic ring are selected from —R₊, —N(R₊)₂, —C(O)R₊, —CO₂R₊, —C(O)C(O)R₊, —C(O)CH₂C(O)R₊, —SO₂R₊, —SO₂N(R₊)₂, —C(═S)N(R₊)₂, —C(═NH)—N(R₊)₂, or —NR₊SO₂R₊; wherein R₊ is hydrogen, an optionally substituted C₁₋₆ aliphatic, optionally substituted phenyl, optionally substituted —O(Ph), optionally substituted —CH₂(Ph), optionally substituted —(CH₂)₁₋₂(Ph); optionally substituted —CH═CH(Ph); or an unsubstituted 5-6 membered heteroaryl or heterocyclic ring having one to four heteroatoms independently selected from oxygen, nitrogen, or sulfur, or, notwithstanding the definition above, two independent occurrences of R₊, on the same substituent or different substituents, taken together with the atom(s) to which each R₊ group is bound, form a 3-8-membered cycloalkyl, heterocyclyl, aryl, or heteroaryl ring having 0-3 heteroatoms independently selected from nitrogen, oxygen, or sulfur. Optional substituents on the aliphatic group or the phenyl ring of R₊ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R₊ is unsubstituted.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ₁₃C- or ₁₄C-enriched carbon are within the scope of this invention. Such compounds are useful, for example, as analytical tools or probes in biological assays.

According to one embodiment, the invention provides a compound having the chemical formula I: 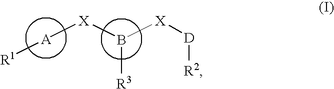
wherein:

A is selected from aryl, or heteroaryl;

B is selected from heterocyclyl, or carbocyclyl and is optionally benzofused;

D is selected from heterocyclyl, carbocyclyl, heteroaryl, or aryl and, when B is benzofused, is additionally selected from —(C₁-C₆)-alkylidene- or —(C₁-C₆)-heteroalkylidene-;

wherein each of A, B and D is optionally additionally substituted with one or more independently selected substitutents;

each X is independently selected from a bond, —C(O)—, —CH₂—, —CH═, or ═CH—;

each or R₁, R₂ and R₃ is independently selected from hydrogen, methyl, or -LS(O)ₘR₄; R₃ is additionally selected from CF₃ or C(O)OR^{o} when B comprises only one substitutable ring atom; and at least one of R₁, R₂ and R₃ is selected from -L-S—CH₃ optionally substituted with halo, O-methyl, O-ethyl, or CN; -L-S—CH₂CH₃ optionally substituted with halo, O-methyl, or CN; -L-S—CH₂CH₂CH₃ optionally substituted with halo; or -L-S—CH(CH₃)CH₃ optionally substituted with halo, wherein:

each m is independently 0, 1 or 2;

each L is independently selected from a bond, an optionally substituted (C₁-C₆)-alkylidene or, an optionally substituted (C₁-C₆)-heteroalkylidene;

R^{o} is selected from hydrogen, optionally substituted C₁₋₆ aliphatic, an unsubstituted carbocyclic ring, an unsubstituted 5-6 membered heteroaryl or heterocyclic ring, an unsubstituted aryl ring, —O-aryl, or —CH₂-aryl, wherein said substituents on the aliphatic group of R^{o} are independently selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of R^{o} is unsubstituted; and

each R₄ is independently selected from hydrogen, aryl, heteroaryl, carbocyclyl, heterocyclyl, —NH—(C₁-C₃)-aliphatic, —NH₂, —O—(C₁-C₃)-aliphatic or —(C₁-C₆)-aliphatic;

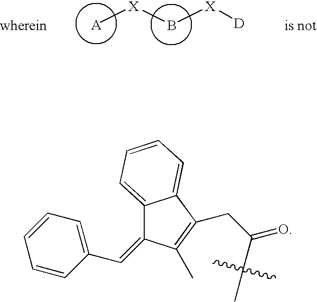

According to one preferred embodiment, ring A is an optionally substituted aryl ring. Even more preferred is when ring A is optionally substituted phenyl. Most preferred is when ring A is a phenyl substituted only at the 4 position with —S(O)₂NH₂, —S(O)CH₃, —SCH₃ or —S(O)₂CH₃.

According to another preferred embodiment, ring B is selected from an optionally substituted heterocyclyl or an optionally substituted benzofused carbocyclyl. More preferably, ring B is an optionally substituted five-membered heterocyclyl or an optionally substituted benzofused five-membered carbocyclyl. Even more preferred is when ring B is selected from 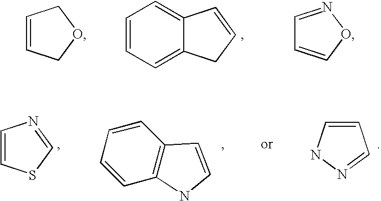
Preferably, the above ring structures have attachments to the rest of the molecule of this invention indicated by -*. 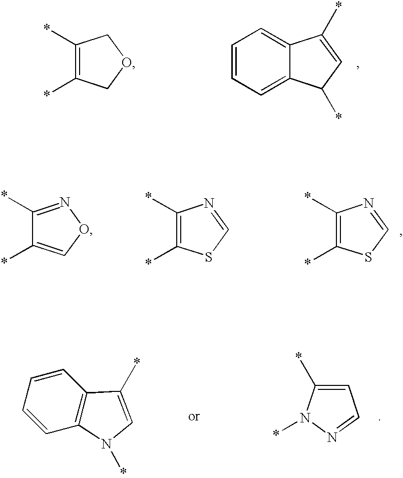
In the most preferred embodiment ring B is selected from the following structures:

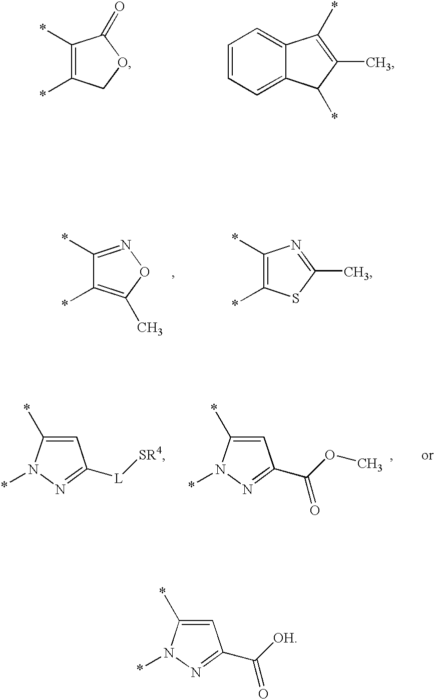

The D component of the compounds of the present invention is preferably selected from optionally substituted aryl or optionally substituted (C₁-C₆)-heteroalkylidene. In a more preferred embodiment, D is selected from optionally substituted phenyl or optionally substituted (C₃-C₆)-heteroalkylidene. Even more preferred is when D is selected from phenyl substituted only at the 4 position with —S(O)₂NH₂, —S(O)CH₃, —SCH₃ or —S(O)₂CH₃, or D is —CH₂C(O)—NH—CH(R₅)—CH₂—CH₂—, wherein R₅ is selected from —C(O)OH or —C(O)OCH₃.

According to one preferred embodiment, when D is a ring structure, each X is a bond.

According to another preferred embodiment, each R₄ is independently selected from hydrogen, —NH—(C₁-C₃)-aliphatic, —NH₂, —O—(C₁-C₃)-aliphatic or —(C₁-C₆)-aliphatic

According to yet another preferred embodiment, at least one of R₁, R₂ and R₃ is selected from -L-S—(C₁-C₃)-straight or branched alkyl. Even more preferred is when at least one of R₁, R₂ and R₃ is selected from -L-S-methyl or -L-S-ethyl. Most preferred is when at least one of R₁, R₂ and R₃ is —S-methyl.

The requirement that at least one of R₁, R₂ and R₃ be selected from -L-S—CH₃ optionally substituted with halo, O-methyl, O-ethyl, or CN; -L-S—CH₂CH₃ optionally substituted with halo, O-methyl, or CN; -L-S—CH₂CH₂CH₃ optionally substituted with halo; or -L-S—CH(CH₃)CH₃ optionally substituted with halo, is because such structures gives the molecule the ability to act as an oxygen scavenger and be regenerated by reduction by MSR It is important to keep the alkyl group on the sulfide small because MSR requires that one group on a corresponding substrate dialkylsuloxide be small, preferably methyl. This has been demonstrated through SAR studies and is evident from examining crystal structures of MSR/substrate complexes [W. T. Lowther et al., Biochemistry, 39, pp. 13307-12 (2000); W. T. Lowther et al., Proc Natl Acad Sci USA, 97, pp. 6463-8 (2000)]. These data indicate that the other alkyl group on the dialkylsulfide can be very large and still serve as a MSR substrate, for example the entire rest of the molecule.

Following oxidation to the corresponding sulfoxide by a free oxygen radical, Msr regenerates the original sulfide structure of this moiety, allowing it to react with and remove another molecule of ROS. This process can continue for many cycles enabling one compound of this invention to react with and inactivate many molecules of ROS. The compounds of this invention are capable of the above-described activity, which is termed “msr-regenerable antioxidant activity.”

In another preferred embodiment, one of R₁ or R₂ is —SCH₃ and the other is —S(O)₂CH₃ or —S(O)₂NH₂.

The proviso that 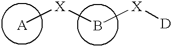
is not 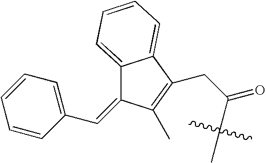
is based upon the fact that the latter core structure is a chemical structure based on or resembling part of the chemical backbone of Sulindac®, a commercially available nonsteroidal anti-inflammatory drug. Catalytic anti-oxidant molecules having (a) a chemical structure based on or resembling part of the chemical backbone structure of Sulindac, (b) at least one methyl sulfoxide moiety and/or a methyl sulfide moiety that can be oxidized to the sulfoxide, and (3) the ability of the methylsulfoxide to serve as a substrate for at least one member of the Msr enzyme family are disclosed in U.S. provisional application Ser. No. 60/429,269. Such compounds are not intended to be part of the present invention.

According to one preferred embodiment, the compounds of the present invention have the formula II: 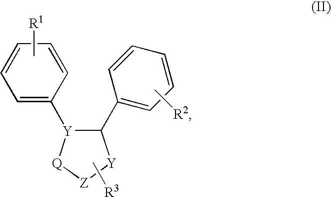
wherein each Y is independently selected from C or N, Q is selected from C, N or S, Z is selected from C or O, and each of R₁, R₂ and R₃ are as defined above.

According to one alternate embodiment, the compound of this invention has the formula (III): 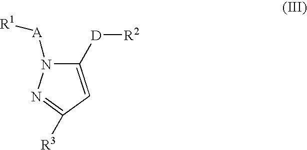
wherein:

each of A and D is aryl;

each of R₁ and R₂ is independently -L-S(O)ₘR₄;

R₃ is selected from:

a) —C(O)—NH—(C₁-C₃)-alkylidene-S—(C₁-C₃)-alkyl, wherein said alkylidene is optionally substituted with one or more halogens and said alkylidene is optionally substituted with —C(O)OH or —C(O)OCH₃, or

b) —S(O)—NH—(C₁-C₃)-alkyl, wherein said alkyl is optionally substituted with one or more halogens;

at least one of R₁, R₂ and R₃ is selected from -L-S—CH₃ optionally substituted with halo, O-methyl, O-ethyl, or CN; -L-S—CH₂CH₃ optionally substituted with halo, Omethyl, or CN; -L-S—CH₂CH₂CH₃ optionally substituted with halo; or -L-S—CH(CH₃)CH₃ optionally substituted with halo, wherein:

each L is independently selected from a bond, an optionally substituted (C₁-C₆)-alkylidene or, an optionally substituted (C₁-C₆)-heteroalkylidene;

each m is independently 0, 1 or 2; and

each R₄ is independently selected from hydrogen, aryl, heteroaryl, carbocyclyl, heterocyclyl, —NH—(C₁-C₃)-aliphatic, —NH₂, —O—(C₁-C₃)-aliphatic, —C(O)—(C₁-C₆)-aliphatic, or —(C₁-C₆)-aliphatic.

For compounds of III, R₃ is preferably —C(O)—NH—CH(R₁₁)—CH₂—S—CH₃, wherein R₁₁ is selected from —COOH, —COOCH₃, —COONH₂, —(C₁-C₆)-aliphatic or —(C₁-C₃)-haloaliphatic.

For a compound of this invention to possess COX-2 inhibitory activity in addition to antioxidant activity, it must comprise a core structure of two aryl rings, preferably phenyl, each attached to a central ring. The two aryl rings must be attached on adjacent positions of the central ring. The central ring is preferably a heterocyclic ring; more preferably a five-membered heterocyclic ring. One of the aryl rings must contain either a sulfonamide or methyl sulfoxide moiety on the 4-position. Substituted sulfonamides or sulfoxides larger than methyl have substantially reduced COX-2 inhibitory activity, as shown by SAR studies in the medicinal chemistry literature [A. S. Kalgutkar et al., Proc Natl Acad Sci USA, 97, pp 925-30 (2000)]. This reduced activity is also supported by published crystal structures of COX-2/inhibitor complexes that show a close interaction of this sulfonamide/sulfoxide group with COX-2 [R. G. Kurumbail et al., Nature, 384, pp 644-648 (1996)]. The second aryl ring may contain small substituents on any position of the ring. However, based upon the prior art and the published crystal structure it is preferred that these additional substituents must be small. Crystal structures of COX-2/inhibitor complexes indicate that substitutions on the 3-position of the central ring, even ones that are bulky, will not interfere with COX-2 inhibitory activity [D. L. Dewitt, Mol. Pharmacol., 55, pp. 526-31(1999)]. In contrast, SAR studies and crystal structures indicate that if a substituent is attached at the 4-position of the central ring, COX-2 inhibitory activity is substantially reduced if not eliminated.

Based upon the above, the antioxidant moiety on molecules having COX-2 inhibitory activity may be located on any position of the second aryl ring or on the 3-position of the central heterocyclyl ring. Compounds of formulae I, II and III that meet the above-described parameters will possess both antioxidant and COX-2 inhibitory activity. Preferably, such dual activity compounds have one of the following structures: 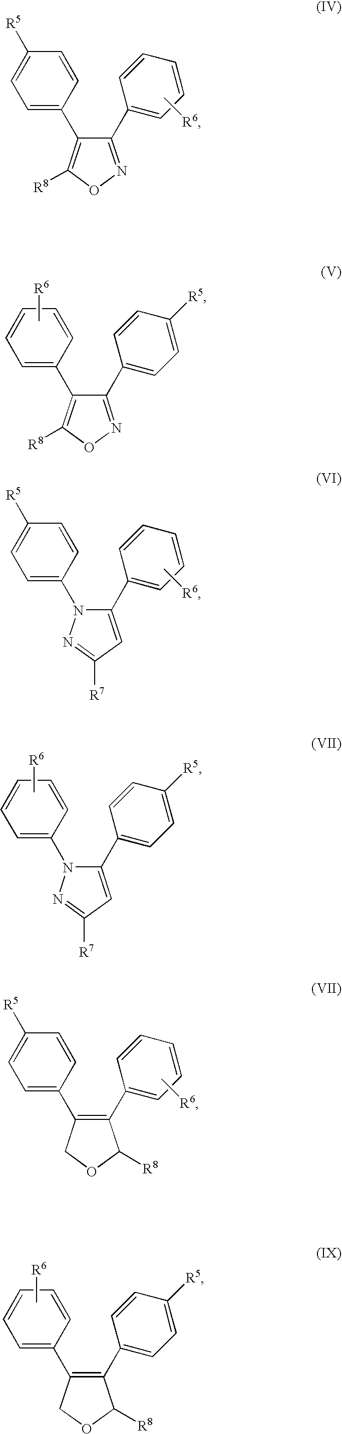 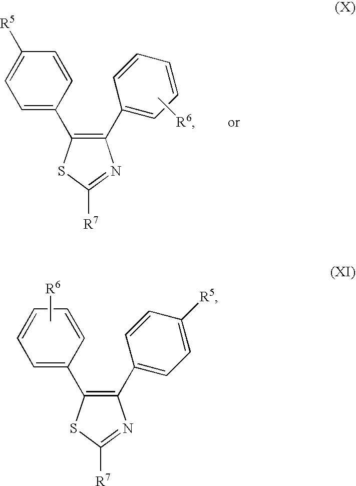
wherein:

R₅ is selected from —S(O)₂NH₂ or —S(O)₂CH₃;

R₆ is selected from —S—CH₃ optionally substituted with halo, O-methyl, O-ethyl, or CN; —S—CH₂CH₃ optionally substituted with halo, O-methyl, or CN; —S—CH₂CH₂CH₃ optionally substituted with halo; or —S—CH(CH₃)CH₃ optionally substituted with halo;

R₇ is selected from hydrogen, -L-S(O)ₘR₄, —C(O)OR^{o}, —CF₃ or —CH₃, wherein R^{o} is selected from hydrogen, optionally substituted C₁₋₆ aliphatic, an unsubstituted carbocyclic ring, an unsubstituted 5-6 membered heteroaryl or heterocyclic ring, an unsubstituted aryl ring, —O-aryl, or —CH₂-aryl, wherein said substituents on the aliphatic group of R^{o} are independently selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of R^{o} is unsubstituted; and

R⁸ is selected from hydrogen, halo, ═O, —CH₃ or —CN.

In some preferred embodiments of compounds of formulae IV-XI, R₅ is —S(O)₂NH₂. In other preferred embodiments of compounds of formulae IV-XI, R₆ is —S—CH₃.

According to a more preferred embodiment, compounds of formula VI have the formula: 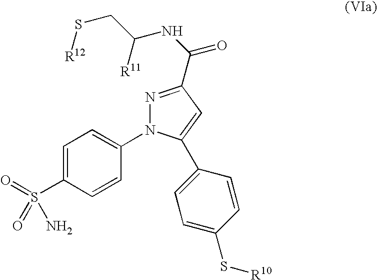
wherein:

each of R₁₀ and R₁₂ is selected from hydrogen, aryl, heteroaryl, carbocyclyl, heterocyclyl, —(C1-C3)-aliphatic-NH2, —(C1-C3)-aliphatic-OH or —(C1-C6)-aliphatic; and

R11 is selected from hydrogen, —COOH, —COOCH3, —COONH2, —(C1-C6)-aliphatic or —(C1-C3)-haloaliphatic,

wherein at least one of R10 and R12 is selected from —CH3 optionally substituted with halo, O-methyl, O-ethyl, or CN; —CH2CH3 optionally substituted with halo, O-methyl, or CN; —CH2CH2CH3 optionally substituted with halo; or —CH(CH3)CH3 optionally substituted with halo.

In another preferred embodiment of formula VI, a compound of formula VIb is provided: 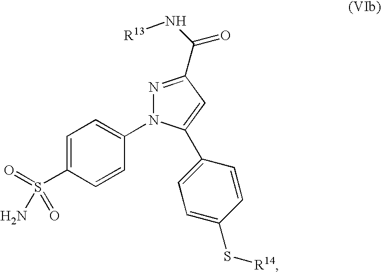
wherein:

R13 is selected from (C1-C6)-aliphatic, an unsubstituted carbocyclic ring, an unsubstituted 5-6 membered heteroaryl or heterocyclic ring, an unsubstituted aryl ring, or -L-S(O)mR4, wherein L, R4 and m are as defined for compounds of formula I; and

R14 is selected from —CH3 optionally substituted with halo, O-methyl, O-ethyl, or CN; —CH2CH3 optionally substituted with halo, O-methyl, or CN; —CH2CH2CH3 optionally substituted with halo; or —CH(CH3)CH3 optionally substituted with halo.

The compounds of formulae IV-XI are based upon the known structure of the COX-2 inhibitors Celebrex® (formulae VI and VII), Valdecoxib® (formula IV, V, X and XI), and Vioxx® (formula VIII and IX). As such, these further preferred compounds possess both Msr-regenerable antioxidant activity and COX-2 inhibitory activity.

Compounds of this invention possessing both COX-2 inhibitory activity and antioxidant activity may be used to treat or prevent oxidative stress, to treat or prevent conditions characterized by undesirable COX-2 activity, or, more preferably, to treat or prevent conditions characterized by both oxidative stress and undesirable COX-2 activity

The following compounds are even more preferred and are representative compounds of the invention:

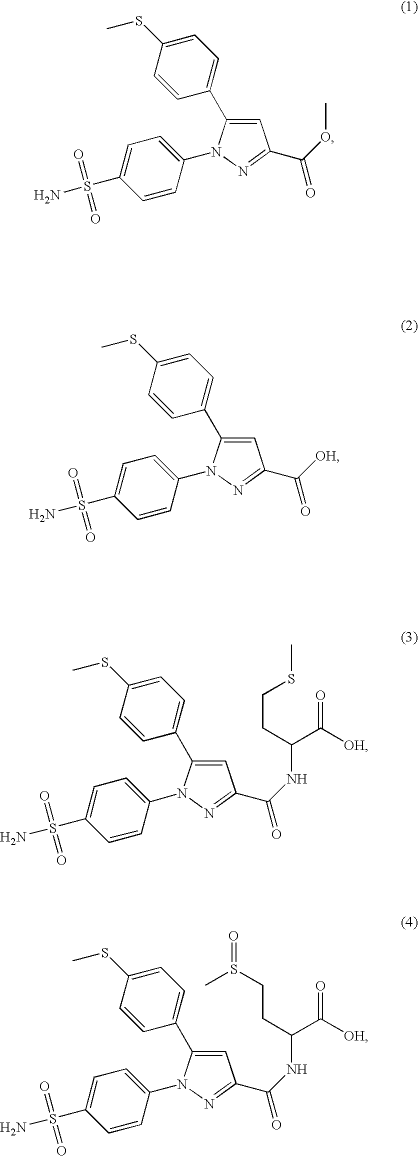

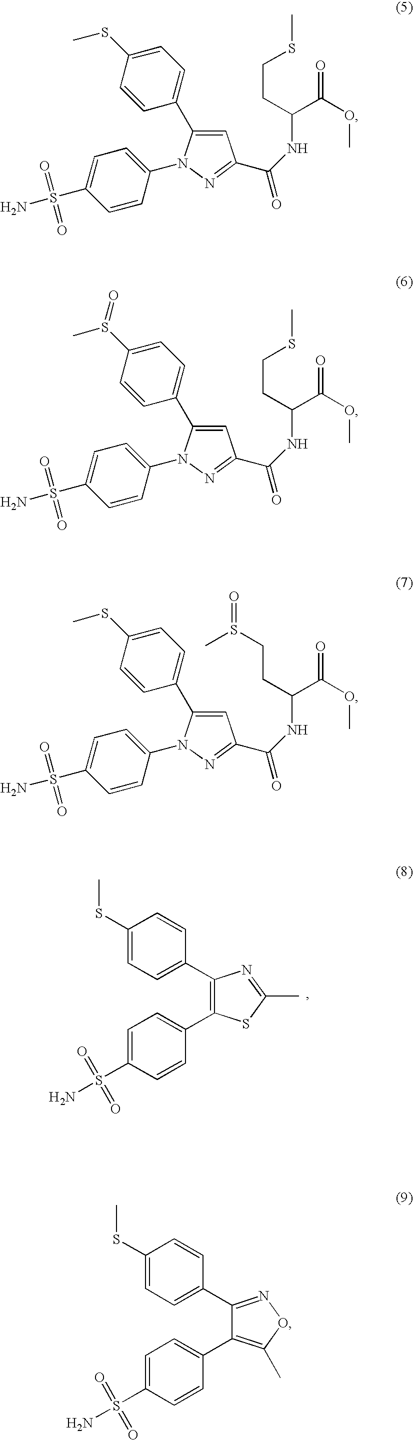

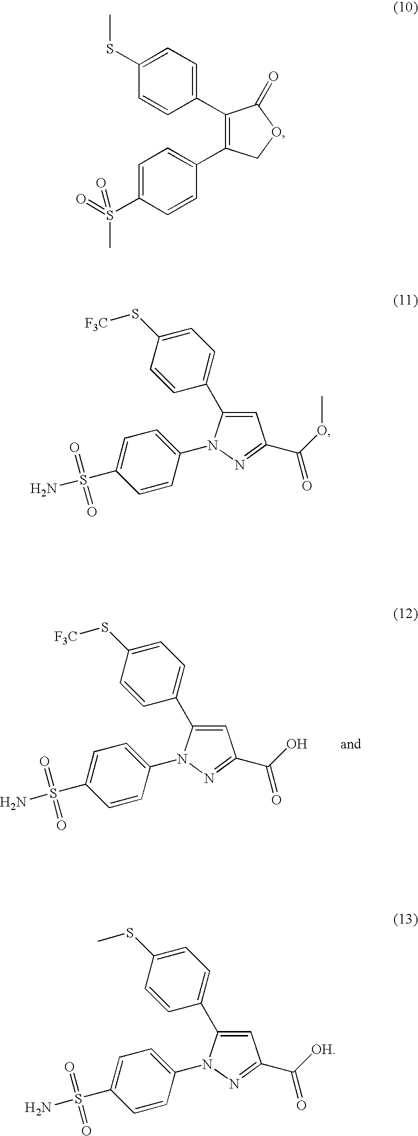

The compounds of this invention can be synthesized from commercially available intermediates by those of ordinary skill in the art, based upon the syntheses set forth in the Examples section.

The compounds of the present invention may be assayed for activity both in vitro and in vivo. For example, in order to assay whether a compound of this invention will serve as a regenerable Msr substrate, the compound is titrated in several increments into a solution of the enzyme msrA (pH 7.5, 37oC, reducing buffer) contained in a microcalorimeter. The heat (ΔH) from the reaction is measured at each step. These power output levels are converted into rates of reaction according to Todd & Gomez, Anal. Biochem., 296, pp. 1-9 (2001), and the resulting rates are plotted versus substrate concentration. From this plot, the kcat and KM are determined according to standard Michaelis-Menton enzyme kinetics.

Alternatively, under the same solution conditions as above, aliquots of enzyme are added to solutions with different concentrations of the potential substrate. After a -21-fixed time has elapsed, the reactions are simultaneously quenched, and the resulting solutions analyzed by HPLC to measure the amount of substrate that has been converted into product. From these data, the initial rate of each reaction can be calculated and plotted vs. substrate concentration as above. The final kcat and KM determination is similar as well.

The compounds of this invention may be tested for antioxidant activity in vivo in Drosophila melanogaster (fruit fly). Flies can be treated with an agent known to produce ROS, such as Paraquat. Test flies are then fed a diet that includes a compound of this invention and their survival is compared to control flies treated with Paraquat, but not subsequently fed a compound of this invention

The compounds of this invention may be tested for their ability to act in the spinal nerve ligation model of neuropathic pain [Kim and Chung (1992) Pain 50:355-363]. Specifically, compounds of this invention may be dosed in rats to investigate the reversal of spinal-ligation induced mechanical allodynia and development of neuropathic pain behavior. It has been observed that systemic injection of a reactive oxygen species scavenger, phenyl-N-tert-butylnitrone (PBN), relieves SNL-induced mechanical allodynia in a dose-dependent manner [Kim et al. (2004) Pain 111:116-124]. Accordingly, compounds of this invention may be tested for their abilities to act at lower effective doses than reference compounds that do not have the ability to regenerate in vivo.

For those compounds of the invention that are expected to also possess COX-2 inhibitory activity, assays well known in the art may be employed. For example, the rat carrageenan foot pad edema test, described in Winter et al., Proc. Soc. Exp. Biol. Med., 111, 544 (1962) and U.S. Pat. No. 5,466,823 may be employed to assess COX-2 inhibitory activity. Alternatively, the rat carrageenan-induced analgesia test, described in Hargreaves et al., Pain, 32, 77 (1988) and in U.S. Pat. No. 5,466,823 may be employed to assess COX-2 inhibitory activity.

The activity of the compounds of this invention against the COX-2 enzyme may also be monitored spectrophotometrically at 590 nm by observing the oxidation of TMPD (N,N,N′,N′-tetramethyl-p-phenylenediamine), as originally detailed by Kulmacz, Arch Biochem Biophys., 249, pp. 273-85 (1986). The reaction is performed with varying amounts of each potential inhibitor, and the extent to which the reaction has proceeded is measured at a fixed time. From these data the inhibition constant (Ki) for each compound is calculated according to standard methods.

Preferred assay methods for both msR activity and COX-2 inhibitory activity, and details thereof are set forth in the Example section.

The compounds of this invention are useful for treating, lessening the severity of or preventing diseases associated with oxidative stress. Such diseases are well-known in the art and include arterial diseases, heart and pulmonary diseases (e.g., myocardial infarction, rheumatoid diseases, eye diseases (e.g., cataracts and macular degeneration), diseases of the gums, respiratory diseases, sickle cell anemia, ischemia, reperfusion injuries, neurodegenerative diseases (e.g., ALS, Alzheimer's Disease, Parkinson's Disease, Huntington's Disease), chronic and acute inflammation, cancer, and reproductive dysfunction. Those compounds of the present invention that also inhibit COX-2 will be particularly effective in treating neurodegenerative diseases, myocardial infarction, stroke, and ischemia.

The compounds of the present invention are useful for the treatment of diseases, disorders, and conditions including, but not limited to acute, chronic, neuropathic, or inflammatory pain, arthritis, migrane, cluster headaches, trigeminal neuralgia, herpetic neuralgia, general neuralgias, epilepsy or epilepsy conditions, neurodegenerative disorders, psychiatric disorders such as anxiety and depression, myotonia, arrythmia, movement disorders, neuroendocrine disorders, ataxia, multiple sclerosis, irritable bowel syndrome, and incontinence. Accordingly, in another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

According to one embodiment, the compounds of the present invention are useful for treating a disease selected from femur cancer pain; non-malignant chronic bone pain; rheumatoid arthritis; osteoarthritis; spinal stenosis; neuropathic low back pain; neuropathic low back pain; myofascial pain syndrome; fibromyalgia; temporomandibular joint pain; chronic visceral pain, including, abdominal; pancreatic; IBS pain; chronic headache pain; migraine; tension headache, including, cluster headaches; chronic neuropathic pain, including, post-herpetic neuralgia; diabetic neuropathy; HIV-associated neuropathy; trigeminal neuralgia; Charcot-Marie Tooth neuropathy; hereditary sensory neuropathies; peripheral nerve injury; painful neuromas; ectopic proximal and distal discharges; radiculopathy; chemotherapy induced neuropathic pain; radiotherapy-induced neuropathic pain; post-mastectomy pain; central pain; spinal cord injury pain; post-stroke pain; thalamic pain; complex regional pain syndrome; phanton pain; intractable pain; acute pain, acute post-operative pain; acute musculoskeletal pain; joint pain; mechanical low back pain; neck pain; tendonitis; injury/exercise pain; acute visceral pain, including, abdominal pain; pyelonephritis; appendicitis; cholecystitis; intestinal obstruction; hernias; etc; chest pain, including, cardiac Pain; pelvic pain, renal colic pain, acute obstetric pain, including, labor pain; cesarean section pain; acute inflammatory, burn and trauma pain; acute intermittent pain, including, endometriosis; acute herpes zoster pain; sickle cell anemia; acute pancreatitis; breakthrough pain; orofacial pain, including, sinusitis pain, dental pain; multiple sclerosis (MS) pain; pain in depression; leprosy pain; behcet's disease pain; adiposis dolorosa; phlebitic pain; Guillain-Barre pain; painful legs and moving toes; Haglund syndrome; erythromelalgia pain; Fabry's disease pain; bladder and urogenital disease, including, urinary incontinence; hyperactivity bladder; painful bladder syndrome; interstitial cyctitis (IC); and prostatitis.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an active metabolite or residue thereof. As used herein, the term “active metabolite or residue thereof” means a metabolite or residue thereof that also possesses msR-regenerable antioxidant activity.

Pharmaceutically acceptable salts are well known in the art. For example, S. M. Berge, et al. describe pharmaceutically acceptable salts in detail in J. Pharmaceutical Sciences, 1977, 66, 1-19, the disclosure of which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N₊(C₁-C₄-alkyl)₄ salts. This invention also envisions the quaternization of any basic nitrogen-containing groups of the compounds disclosed herein. Water or oilsoluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, lower alkyl sulfonate and aryl sulfonate.

The compounds of the present invention (including pharmaceutically acceptable derivatives thereof) may be formulated into compositions and, in particular, pharmaceutically acceptable compositions for administration to a mammal for the treatment, amelioration or prevention of a disease or condition associated with oxidative stress. The compositions of this invention comprise any of the compounds as described herein in an amount effective to reduce oxidative stress, and optionally comprise a pharmaceutically acceptable carrier, adjuvant or vehicle. The term “effective to reduce oxidative stress,” as used herein means lessening the severity of a disease or condition caused by oxidative stress and/or detectably reducing the amount of ROS in a biological sample or in a mammal. In certain embodiments, these compositions optionally further comprise one or more additional therapeutic agents.

In yet another aspect, a method for the treatment or lessening the severity of acute, chronic, neuropathic, or inflammatory pain, arthritis, migrane, cluster headaches, trigeminal neuralgia, herpetic neuralgia, general neuralgias, epilepsy or epilepsy conditions, neurodegenerative disorders, psychiatric disorders such as anxiety and depression, myotonia, arrythmia, movement disorders, neuroendocrine disorders, ataxia, multiple sclerosis, irritable bowel syndrome, incontinence, visceral pain, osteoarthritis pain, postherpetic neuralgia, diabetic neuropathy, radicular pain, sciatica, back pain, head or neck pain, severe or intractable pain, nociceptive pain, breakthrough pain, postsurgical pain, or cancer pain is provided comprising administering an effective amount of a compound, or a pharmaceutically acceptable composition comprising a compound to a subject in need thereof. In certain embodiments, a method for the treatment or lessening the severity of acute, chronic, neuropathic, or inflammatory pain is provided comprising administering an effective amount of a compound or a pharmaceutically acceptable composition to a subject in need thereof. In certain other embodiments, a method for the treatment or lessening the severity of radicular pain, sciatica, back pain, head pain, or neck pain is provided comprising administering an effective amount of a compound or a pharmaceutically acceptable composition to a subject in need thereof. In still other embodiments, a method for the treatment or lessening the severity of severe or intractable pain, acute pain, postsurgical pain, back pain, or cancer pain is provided comprising administering an effective amount of a compound or a pharmaceutically acceptable composition to a subject in need thereof.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

According to one embodiment, the invention provides a method of treating, lessening the severity or preventing a disease or condition caused or exacerbated by oxidative stress. The term “caused or exacerbated by oxidative stress” as used herein means a disease or condition in which oxidative stress is known or suspected of playing a direct or indirect role. This method comprises the step of administering to a mammal an amount of a compound or composition according to the present invention effective to reduce or prevent oxidative damage to cells in said mammal. Amounts effective to reduce or prevent oxidative damage to cells in a mammal will range from about 0.1-100 mg/kg of body weight one or more times a day. More preferably, the amount will range from about 1-25 mg/kg of body weight one or more times a day. However, it will be understood by the person of ordinary skill in the art, given the benefit of this disclosure, that the specific dose level for any particular mammal will depend upon a variety of factors including but not limited to the species being treated, the disorder being treated and the severity of the disorder; the activity of the specific compound employed; the specific composition employed; the age, body weight, general health, sex and diet of the mammal; the time of administration, route of administration, and rate of excretion of the specific compound employed; the duration of the treatment; drugs used in combination or coincidental with the specific compound employed, and like factors well known in the medical arts.

In one preferred embodiment, the condition to be treated is selected from arterial diseases, heart and pulmonary diseases (e.g., myocardial infarction, rheumatoid diseases, eye diseases (e.g., cataracts and macular degeneration), diseases of the gums, respiratory diseases, sickle cell anemia, ischemia, reperfusion injuries, neurodegenerative diseases (e.g., ALS, Alzheimer's Disease, Parkinson's Disease, Huntington's Disease), or chronic and acute inflammation.

According to another embodiment, the invention provides a method of treating, lessening the severity or preventing a disease or condition caused or exacerbated by both oxidative stress and inflammation. This method comprises the step of administering to a mammal a compound of or composition according to the present invention that possesses both msr-regenerable antioxidant and COX-2 inhibitory activities. Preferably, such active compounds are selected from compounds of any of formulae IV-XI, above. In a preferred embodiment, this method is employed to treat, lessen the severity of, or prevent neurodegenerative diseases, myocardial infarction, stroke, or ischemia.

When utilized in the methods of this invention, the compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment.

The pharmaceutically acceptable compositions of this invention can be administered to humans and other mammals orally, rectally, parenterally, intracisternally, intravaginally, intraperitoneally, topically (as by powders, ointments, or drops), bucally, as an oral or nasal spray, or the like, depending on the severity of the infection being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable nonirritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to another embodiment, the compounds of this invention may be co-administered with an active agent other than a compound of this invention that is normally used in the treatment of the disease or condition characterized by oxidative stress. The choice of additional agent will be based upon the disease or condition to be treated and the judgment of the treating physician. The amount of the additional active agent will be equal to or less than the amount utilized when that agent is administered in a monotherapy. Preferably, that agent will be present in the compositions of this invention at between 10-100% of the monotherapy dose. More preferably the agent will be present at between 50-100% of the monotherapy dose.

The additional agents which may be present in the compositions of the present invention include, but are not limited to, agents used in the treatment of arterial disease, heart and pulmonary diseases, rheumatoid disease, eye disease (cataract, macular degeneration), gum disease, respiratory disease, Sickle Cell Anemia, ischemia/reperfusion injuries, neurodegenerative diseases (ALS, Alzheimer's Disease, Huntington's disease), inflammation (chronic and acute), cancer, and reproductive dysfunction.

Examples of additional agents useful to treat inflammation include, but are not limited to, aceclofenac, acemetacin, e-acetamidocaproic acid, acetaminosalol, 21-acetoxypregnenolone, alclofenac, alclometasone, algestone, alminoprofen, amfenac, 3-amino-4-hydroxybutyric acid, amiprilose, ampiroxicam, amtolmetin guacil, apazone, aspirin, balsalazide, beclomethasone, bendazac, benorylate, benoxaprofen, benzpiperylon, benzydamine, bermoprofen, a-bisabolol, bromelain, bromfenac, bucolome, budesonide, bufexamac, bumadizon, butibufen, calcium acetylsalicylate, calendula, carprofen, celecoxib, chamomile, cinmetacin, clidanac, clobetasol, clobetasone, clopirac, deflazacort, desonide, desoximetasone, dexamethasone, diclofenac, difenamizole, difenpiramide, diflorasone, diflucortolone, diflunisal, difluprednate, ditazol, droxicam, emorfazone, enfenamic acid, enoxolone, epirizole, etanercept, etodolac, etofenamate, felbinac, fenbufen, fenclozic acid, fendosal, fenoprofen, fentiazac, fepradinol, feprazone, fluazacort, flufenamic acid, flumethasone, flunoxaprofen, fluocinolone acetonide, fluocinonide, fluocortin butyl, fluorometholone, fluperolone acetate, fluprednidene acetate, fluprednisolone, flurandrenolide, flurbiprofen, fluticasone propionate, gentisic acid, glucametacin, glycol salicylate, guaiazulene, halcinonide, halobetasol propionate, halometasone, halopredone acetate, ibufenac, ibuprofen, ibuproxam, imidazole salicylate, indomethacin, indoprofen, infliximab, interleukin-10, interleukin-1 receptor agonist, isonixin, isoxepac, isoxicam, ketoprofen, ketorolac, lexipafant, lonazolac, lornoxicam, loteprednol etabonate, loxoprofen, lysine acetylsalicylate, mazipredone, meclofenamic acid, mefenamic acid, meloxicam, mesalamine, metiazinic acid, mofebutazone, mofezolac, mometasone furoate, morazone, morpholine, nabumetone, 1-napthyl salicylate, naproxen, niflumic acid, nimesulide, olsalazine, oxaceprol, oxametacine, oxaprozin, oxyphenbutazone, paranyline, parsalmide, perisoxal, phenyl acetylsalicylate, phenylbutazone, phenyl salicylate, piketoprofen, pipebuzone, pirazolac, piroxicam, pirprofen, pranoprofen, prednisone, proglumetacin, propyphenazone, proquazone, protizinic acid, remifenazone, rimexolone, rofecoxib, salacetamide, salicylamide O-acetic acid, salicylic acid, salicylsulfuric acid, salsalate, sodium chloride, sulfasalazine, sulindac, superoxide dismutase, suprofen, suxibuzone, talniflumate, tenidap, tenoxicam, terofenamate, thiazolinobutazone, tiaprofenic acid, tiaramide, tinoridine, tixocortol, tolfenamic acid, tolmetin, triamcinolone benetonide, triamcinolone hexacetonide, tropesin, xenbucin, ximoprofen, zaltoprofen, and zomepirac.

Examples of agents useful to treated rheumatoid disease include, but are not limited to, S-adenosylmethionine, auranofin, aurothioglucose, azathioprine, bucillamine, chloroquine, cuproxoline, gold sodium thiomalate, gold sodium thiosulfate, hydroxychloroquine, kebuzone, leflunomide, lobenzarit, melittin, methotrexate, penicillamine, phytolacca, and savin.

Examples of agents useful to treat arterial heart and pulmonary diseases include, but are not limited to, acifran, acipimox, alminum nicotinate, atorvastatin, benfluorex, bbenzalbutyramide, benzafibrate, binifibrate, carnitine, cerivastatin, cholestyramine resin, chondroitin sulfate, ciprofibrate, clinofibrate, clofibrate, clofibric acid, clomestrone, colesevelam hydrochloride, colestipol, dexatran, dexatran sulfate sodium, eicosapentaenoic acid, etiroxate, etofibrate, ezetimibe, fenofibrate, fluvastatin, gemfibrozil, lovastatin, meglutol, melinamide, niceritol, nicotinic acid, y-oryzanol, oxiniacic acid, pantethine, pirfibrate, pirozadil, pravastatin sodium, probucol, 3-pyridineacetic acid, ronifibrate, sultosilic acid, theofibrate, thyropropic acid, thyroxine, tiadenol, xenbucin, acadesine, cariporide, phosphocreatine, acebutolol, alprenolol, amlodipine, arotinolol, atenolol, bepridil, bevantolol, bucumolol, bufetolol, bufuralol, bunitrolol, bupranolol, carazolol, carteolol, celiprolol, cinepazet, diltiazem, elgodipine, felodipine, gallopamil, imolamine, indenolol, isosorbide dinitrate, isradipine, limaprost, mepindolol, metoprolol, molsidomine, nadolol, nicardipine, nicorandil, nifedipine, nifenalol, nilvadipine, nipradilol, nisoldipine, nitroglycerin, oxprenolol, oxyfedrine, ozagrel, penbutolol, pindolol, pronethalol, propranolol, ranolazine, semotiadil, sotalol, tedisamil, terodiline, timolol, toliprolol, trimetazidine, trolnitrate phosphate, verapamil, and zatebradine.

Examples of agents useful to treat neurodegenerative diseases include, but are not limited to, amantadine, apomorphine, benserazide, benzetimide, benztropine mesylate, bietanautine, biperiden, bromocriptine, budipine, cabergoline, carbidopa, dexetimide, diethazine, droxidopa, entacapone, ethopropazine, ethylbenztropine, idazoxan, lazabemide, levodopa, lisuride, memantine, methixene, mofegiline, pergolide, phenglutarimide, piroheptine, pramipexole, pridinol, procyclidine, prodipine, ropinirole, scopolamine N-oxide, selegiline, talipexole, terguride, tolcapone, trihexiphenidyl hydrochloride, tropacine, aptiganel, citicoline, dexanabinol, ebselen, licostinel, lubeluzole, remacemide, repinotan, riluzole, xaliproden, and ziconotide.

Examples of agents useful to treat ischemia/reperfusion injuries include, but are not limited to, aceglutamide, acetylcamitine, aniracetam, besipirdine, bifemelane, choline alfoscerate, donepezil, exifone, fipexide, ginkgo, idebenone, indeloxazine hydrochloride, ipidacrine, leteprinim, nebracetam, nefiracetam, nizofenone, oxiracetam, piracetam, posatirelin, pramiracetam, propentofylline, pyritinol, rivastigmine, sabcomeline, sabeluzole, tacrine, velnacrine, vinconate, and xanomeline.

The compounds of this invention may also be administered with other compounds known to be useful in the treatment of pain. For example, exemplary additional therapeutic agents include, but are not limited to: nonopioid analgesics (indoles such as Etodolac, Indomethacin, Sulindac, Tolmetin; naphthylalkanones such as Nabumetone; oxicams such as Piroxicam; para-aminophenol derivatives, such as Acetaminophen; propionic acids such as Fenoprofen, Flurbiprofen, Ibuprofen, Ketoprofen, Naproxen, Naproxen sodium, Oxaprozin; salicylates such as Asprin, Choline magnesium trisalicylate, Diflunisal; fenamates such as meclofenamic acid, Mefenamic acid; and pyrazoles such as Phenylbutazone); or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blocade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Seventeenth Edition, Ed. Mark H. Beers and Robert Berkow, Merck Research Laboratories, 1999, and the Food and Drug Administration website, www.fda.gov, the entire contents of which are hereby incorporated by reference.

These additional agents may be administered separately, as part of a multiple dosage regimen, from the msr-regenerable antioxidant-containing composition. Separate administration may be carried out by administering the additional agent prior to, simultaneously with of following administration of a compound of this invention. Alternatively, these additional agents may be part of a single dosage form, mixed together with the msr-regenerable antioxidant in a single composition.

According to yet another embodiment, the compounds of this invention may be incorporated into compositions for coating an implantable medical device, such as prostheses, artificial valves, vascular grafts, stents and catheters. Suitable coatings and the general preparation of coated implantable devices are described in U.S. Pat. Nos. 6,099,562; 5,886,026; and 5,304,121, the disclosures of which are herein incorporated by reference. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provide a method of coating an implantable medical device comprising the step of contacting said device with the coating composition described above. It will be obvious to those skilled in the art that the coating of the device will occur prior to implantation into a mammal.

According to another embodiment, the invention provides a method of inhibiting, reducing, or preventing oxidative damage in a biological sample. This method comprises the step of contacting said biological sample with a compound of this invention. The term “biological sample,” as used herein includes cell cultures or extracts thereof; biopsied material obtained from a mammal or extracts thereof; and blood, saliva, urine, feces, semen, tears, or other body fluids or extracts thereof.

In order that the invention described herein may be more fully understood, the following examples are set forth. It should be understood that these examples are for illustrative purposes only and are not to be construed as limiting this invention in any manner.

### EXAMPLE 1

### Assays

Methionine Sulfoxide Reductase Assay. An assay using isothermal titration calorimetry was developed. This assay was used to evaluate compounds as substrates for methionine sulfoxide reductase (msrA). Compounds that are readily reduced by this enzyme are potentially good scavengers of reactive oxygen species (ROS). The quicker such compounds are converted back to their reduced form by MSR, the more equivalents of ROS they will eliminate before being cleared from the body. The assay was used to determine the enzymatic parameters (k_{cat} and K_{M}) of the compounds of this invention interacting with msrA under standard conditions. Chemical modifications that increase k_{cat}, a measure of how quickly a compound was turned over by the enzyme, and/or decrease its K_{M}, a measure of how readily a compound bound to the enzyme, represent improvements in that compound's ability to be efficiently processed by msrA.

In order to run the assay bovine msrA was overexpressed in E. coli and purified according to the method described in Lowther et al., Proc. Natl. Acad. Sci. USA, 97, pp. 6463-6468 (2000), the disclosure of which is herein incorporated by reference. A 300 nM solution of the enzyme was prepared in 50 mM Tris, 50 mM KCl, 4 mM tris(carboxyethyl)phosphine (TCEP), pH 7.50. A 25 mM solution of the compound to be tested was prepared in the identical buffer. A 1.8 ml aliquot of the msrA enzyme solution was loaded into the cell of a VP-ITC instrument from Microcal Corp. (Northampton, Mass.). The test compound solution was loaded into the syringe, and the entire system was allowed to come to thermal equilibrium at 37° C. while stirring at a rate of 300 rpm. Once equilibrium was established, a series of small injections (1-20 ml) of the compound into the protein solution were made, and the increasing power output of the enzyme was measured after each injection, relative to the instrument baseline. These power output levels were converted into rates of reaction, according to Todd & Gomez, Anal. Biochem., 296, pp. 1-9 (2001), the disclosure of which is herein incorporated, and the resulting rates were plotted against substrate concentration. From this plot, the k_{cat} and K_{M} were determined with a computer fitting routine according to standard Michaelis-Menton enzyme kinetics.

We have also developed an HPLC assay for MSR enzymatic activity. The BPLC assay was carried out in the same solution conditions as the calorimetric assay above (50 mM Tris, 50 mM KCl, 4 mM TCEP, pH 7.50). Twenty 100 ml aliquots of the compound to be assayed were prepared in concentrations ranging from 1 mM to 20 mM. To each of these solutions we added 20 ml of a 100 mM solution of msrA (final volume 120 ml). After 30 minutes, each reaction was quenched by the addition of 200 ml of acetonitrile. Solutions were then centrifuged for 5 minutes (14,000 rpm in an Eppendorf Microcentrifuge), and 10 ml of the clear supernatant from each tube was individually injected onto a 10 cm C18 column (Apex, Jones Chromatography, Denver, Colo.) equilibrated at 50° C. in buffer containing 30% acetonitrile. Using a linear gradient from 30 to 70% acetonitrile over 11 minutes, the reduced and oxidized forms of the compound were eluted. The oxidized form of the compound eluted first and in a distinct peak from the reduced form. The relative peak intensities measured at 250 nm allowed us to determine the initial rate of reaction for each substrate concentration, using the calculations shown by Moskovitz et al., Methods Enzymol., 300, pp. 239-244 (1999). From these data, the final k_{cat} and K_{M} fitting produced similar results as that determined in the calorimetric assay described above.

COX-2 Assay. In order to identify compounds that are also potent inhibitors of cyclooxygenase-2 we assayed their ability to inhibit the reaction of COX-2 with standard substrates. The activity of the COX-2 enzyme was monitored spectrophotometrically at 590 nm by observing the oxidation of TMPD (N,N,N′,N′-tetramethyl-pphenylenediamine), as originally detailed by Kulmacz, Arch Biochem Biophys., 249, pp. 273-85 (1986), the disclosure of which is herein incorporated by reference. The reaction was performed with varying amounts of each test compound, and the extent to which the reaction proceeded was measured at a fixed time. From these data the inhibition constant (Kᵢ) for each compound was calculated according to standard methods.

MPTP Assay. Test animals are administered MPTP (either a single dosage of 30 mg/kg; two dosages of 30 mg/kg separated by a 16 hour interval or four dosage of 15-20 mg/kg separated by 2 hour intervals) in phosphate buffered saline (“PBS”), pH 7.4, either intraperitoneally or subcutaneously. Control animals (no MPTP) are given PBS. A compound of this invention is administered intravenously to the animals at various doses (0-100 mg/kg), either 10 minutes before administration of MPTP or some time after administration.

MPTP-induced markers of degeneration include striatal dopamine depletion, locomotive activity decrease, cell loss, and tyrosine hydroxylase immunoreactivity (increased tyrosine levels=increased oxidative damage). Two to four minutes following an injection of 30 mg/kg MPTP intraperitoneally, mice exhibit physical signs of Parkinsonism, such as serotonin syndromes characterized by splayed hind limb, tremor, and straub tail. After 3 hours, dopamine-mediated behavior, such as akinesia and rigidity is observed. After 7 days, mice are subjected to a swim test to measure general coordination. For tissue and organ assays, animals are sacrificed 7 days after initial injection.

Tremor and hind limb abduction in mice is scored for intensity on a scale of 0-4. Akinesia is measured by noting the latency of the animals to move all four limbs in a unit of time. Catalepsy is measured by placing the animals on a flat horizontal surface with both hind limbs on a square wooden block (3 cm) and then latency (in seconds) required to move the hind limbs from the block to the ground is measured. Swim tests were conducted in water tubs and scored based as follows: 0, hind part of animal sinks with head floating; 1, occasional swimming using hind limbs while floating on one side; 2, occasional floating/swimming only; 3, continuous swimming. General locomotive activity is measured using activity cages, one per animal, equipped with photobeams to monitor animal movements. Computer programs connected to the cages measure movements at 1 minute intervals for 60 minutes following injection of both MPTP and a compound of this invention and the measurements are repeated 1 day later.

An effective dose of a compound of this will prevent, delay, or reduce the severity of these indications, as measured by standard scoring procedures for these types of locomotive tests. Dosages that prevent these conditions are preferred. When a compound of this invention is administered after MPTP injection, improvement of Parkinsonian symptoms will be observed.

Whole Blood Assay. Blood samples are taken from animals having an indication involving oxidative stress after showing signs of that indication. The animal is then treated with a compound of the invention and additional blood samples are taken at various time points following treatment. The blood samples, or polymorphonuclear neutrophils isolated from the samples are then incubated for 15 minutes with 2′,7′-DCFH-DA (2′,7′-dichlorofluorescin-diaceate) (100 μmol/L or 5 μmol/L) at 37° C. DCFH-DA diffuses into the cells and is hydrolyzed into 2′7′-DCFH, which is nonfluorescent, but is readily oxidized by H2O2 to highly fluorescent DCF in the presence of peroxidase. Flow cytometry analysis is carried out under standard conditions to separate the green fluorescence of DCF (515-545 nm). Collection range on the instrument can be set to between 500 and 560 nm (530 nm±30). Blood cells isolated from an animal having an indication involving an oxidative stress following treatment with a compound of this invention will exhibit lower fluorescence than untreated animals having the same indication.

Alternatively, blood cells from healthy animals are stimulated to produce H2O2 by addition of TNF (tumor necrosis factor)-alpha combined with FMLP, similar to the process described for MPTP. This procedure stimulates production of oxidative stress, mimicking that involved in Parkinson's and other neurodegenerative diseases and inflammatory conditions such as ALS, Alzheimer's, atherosclerosis, and gout. The cells are then treated with various amounts of a compound of this invention for various times. following treatment, the cells are incubated with 2′,7′-DCFH-DA, as described above, and subjected to flow cytometry analysis.

PC-12 Cell Assay: The assay is run in 96-well plates, and each well is filled with 0.1 ml of cell solution containing approximately 2×104 cells/well. A compound of this invention is added to the wells in doses ranging from 0-200 μM, and then H2O2 is added in doses ranging from 0-400 μM. Cells are then incubated at 37° C. for 2 hours. After that time, cell viability is determined by transferring into a medium containing exactly the same components as when they were treated with compound and H2O2, as well as 5 mg/ml of 3-(4,5-dimethylthiazol-2-yl)-2,5-diphenyltetrazolium bromide (“MTT”). MTT is metabolized by active mitochondria in healthy cells inducing a color change (yellow to deep brown), which is monitored at 570 nm. Accumulation of the deep brown color (formazan) is directly proportional to cell viability. Each cell solution is monitored after 2 and 4 hours. Cells treated with compound will demonstrate a significantly greater increase in absorbance at 570 nm, as compared to control cells treated with no compound.

Spinal Nerve Ligation Model: Rats are anesthetized with halethane in oxygen and then the L5 spinal nerve is tightly ligated using 6-0 silk thereat [Kim and Chung (1992) Pain 50:355-363]. The wound is treated with 10% providone iodine solution and closed with wound clips. Behavioral tests measuring foot withdrawal thresholds in response to mechanical stimuli applied to the left hind paw are executed. For each test, the animal is placed in plastic chamber (8.5×8.5×28 cm) and habituated for 15 minutes. The chamber is placed on top of a mesh screen so that mechanical stimuli could be administered to the plantar surface behind the left hind paw. Thresholds are determined using a set of von Frey monofilaments [Kim et al. (2004) Pain 111:116-124].

### EXAMPLE 2

### General Principles for the Design of Compounds

We began the design of the compounds of the present invention by choosing scaffolds based upon existing COX-2 inhibitors and that could cross the blood brain barrier. We next analyzed those scaffolds for sites where derivatizations could be made in order to produce compounds that contained moieties that could become oxidized by reaction with ROS and then reduced by the action of MSR without interfering with COX-2 inhibitory activity.

To ensure that the compounds would be substrates for MSR, we determined the substrate specificities of MSR by choosing test compounds that contained a sulfoxide moiety and testing those compounds for MSR activity in the enzyme assay described below. Table 1 sets forth compounds that were tested for their ability to serve as MSR substrates using the isothermal titration calorimetry assay described above:

TABLE 1
Assay of ability of compounds to act as a substrate for msrA.

| | K_{cat} | |
|---|---|---|
| Compound | (sec⁻¹) | K_{M} (mM) |
| methionine sulfoxide (compound 100) | 0.27 | 0.504 |
| 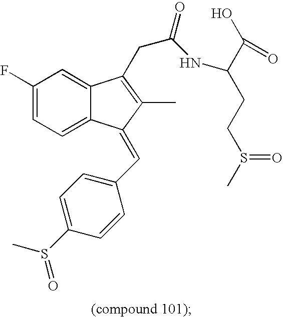 | 0.27 | .0076 |
| SYCB-102 | | |
| methyphenylsulfoxide (compound 102) | 0.46 | 0.750 |
| DMSO (compound 103) | 0.064 | 0.314 |
| Sulindac (compound 104) | 0.56 | 0.112 |
| 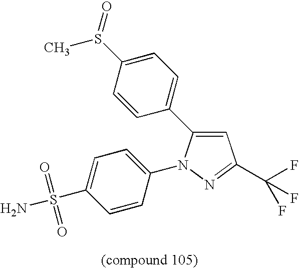 | ND | ND |
| 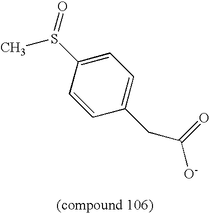 | ND | ND |
| 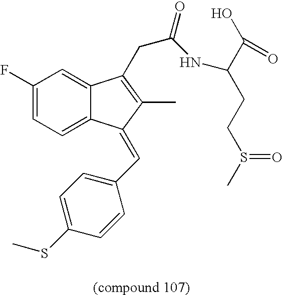 | ND | ND |
| 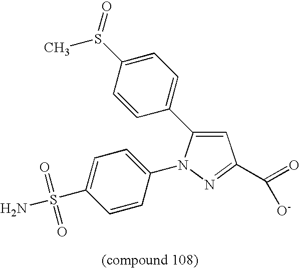 | ND | ND |
| methionine sulfone (compound 109) | 0 | 0 |

ND = not determined.

Compounds 105-108 in the list were only soluble below 1 mM. However, an assessment was made as to whether those compounds were substrates for msrA (i.e., whether they are turned over by the enzyme), by employing the following experimental protocol. The compounds were dissolved in the usual buffer [50 mM Tris, 50 mM KCl, 5 mM TCEP, pH 7.50] at a concentration of 500 μM, or as high as possible. 1.8 ml of this solution was loaded into the calorimetric cell, while the syringe was filled with a 100 mM solution of msrA in the same buffer. After the stirring and equilibration period has elapsed, three injections of protein were made, spaced out over 20 minute intervals. If the compound is a substrate for msrA, a large amount of heat will be evolved after the first injection (several millicalories generated over ˜10 minutes), while the other two injections of protein will register only small heats (tens of microcalories), before the signal quickly returns to the baseline. These smaller heats represent the heat of dilution of the protein into the solution in the calorimetric cell. If there is no reaction between the enzyme and the compound (meaning the compound is not a substrate for msrA), each injection will generate only the heat of dilution—all three injections will look identical. Each of compounds 105 through 108 was shown to be substrates for msrA by this method. Finally, compound 109 (methionine sulfone) was tested as a negative control in this assay and we confirmed that it does not get turned over by msrA.

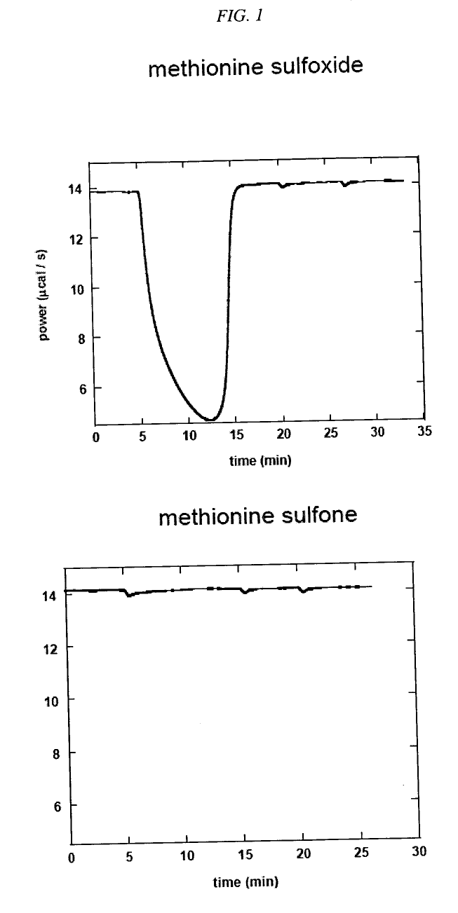

FIG. 1 shows two calorimetric experiments in which a solution of msrA is injected into solutions of methionine sulfoxide and methionine sulfone. When the enzyme reacts with methionine sulfoxide, it produces the large heat output seen after the first arrow. Once the substrate has been fully reacted and the heat output has returned to the baseline, further injections of protein show only the heat of dilution of the enzyme. For methionine sulfone, which is not a substrate for msrA, all three injections of the protein give only the heat of dilution.

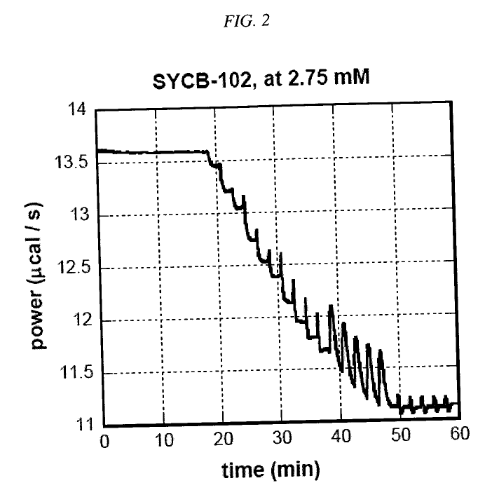

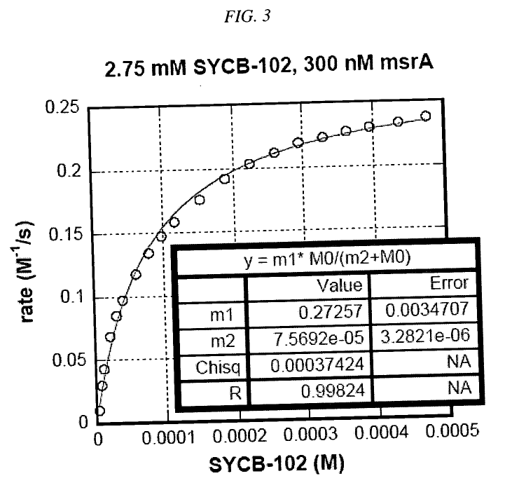

The assay data for compound 101 is shown in FIG. 2. We used a 2.75 mM solution of compound 101 in the injection syringe. For other compounds we used up to a 25 mM solution. After the stirring and equilibration (typically 20 min.), the baseline heat output for the experiment was established, and then a series of injections of the compound were made into the protein solution. Each injection increased the concentration of substrate in the calorimetric cell, thus further saturating the enzyme and increasing the rate of turnover, as shown by the changing levels of heat output in FIG. 2 below: FIG. 3 demonstrates the rate of the reaction versus varying concentrations of compound 101 and the calculations of Kcat and KM.

We next determined how to link the reduced version of those test structures that could serve as an MSR substrate or some variant thereof to the COX-2 inhibitor scaffolds.

In order to design compounds that additionally inhibited COX-2, we modeled structures of compounds known to inhibit COX-2 and derivatized to contain an MSRregeneratable substrate bound to the active site of COX-2 by utilizing the coordinates of the crystal structures of COX-2.

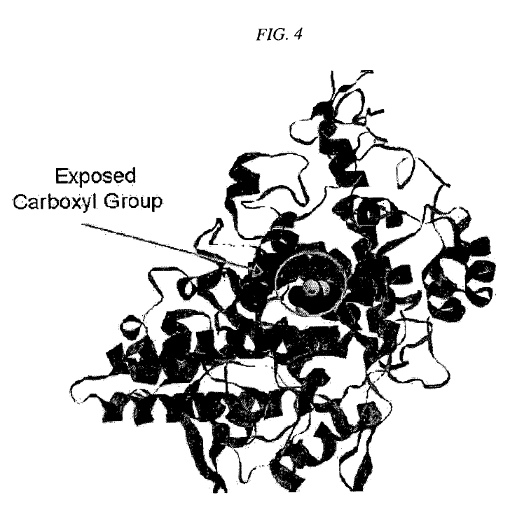

To test the ability of compounds to act as multi-functional compounds, calculations were performed on a crystal structure of COX-2 bound to indomethacin [Kurumbail et al., Nature, 384, pp. 644-8 (1996)]. Surface area calculations were performed to identify portions of the ligand that remain accessible to solvent when the ligand is bound to protein. The calculations were performed using the GETAREA program, available at http://www.scsb.utmb.edu/getarea. The probe radius used in the calculation was 1.4 Angstroms. The results indicated that only the O3 carboxyl oxygen of indomethacin shows any substantial solvent exposure when the molecule is bound to COX-2 (see FIG. 4). A large loop of three helices, consisting of residues 86-122 (shown in dark gray in FIG. 4) forms a channel through which the carboxyl moiety of indomethacin is accessible to the solvent. It is possible to design certain compounds that bind to COX-2 and have other groups that are available to react with other species in situ.

Based on the modeled structures, we designed compounds for which we could conceive synthetic routes that would theoretically not interfere with the COX-2 selective inhibition properties, and that would become oxidized by ROS, then reduced by MSR. We preferably aimed to achieve compounds that would be orally available and that would cross the blood brain barrier. Following synthesis, we tested the compounds for their ability to inhibit COX-2 and for their abilities to be substrates for MSR.

### EXAMPLE 3

### Synthesis of Compound 3

We synthesized compound 3 according to the following scheme:

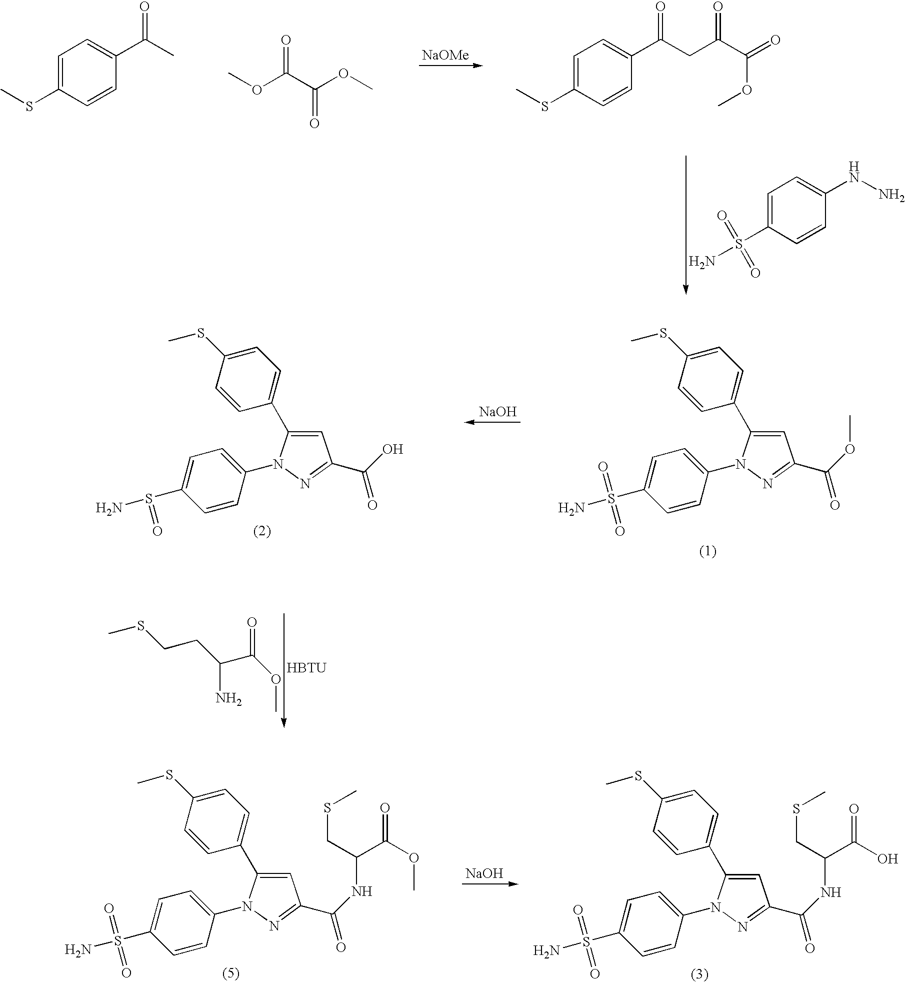

We mixed a solution of 400 mg of dimethyl oxalate in 10 ml of methyl-t-butyl ether (MTBE) with a solution of 100 mg of sodium metal dissolved in 1.6 ml of methanol. To the above mixture, we added a solution of 0.5 g of 4-thiomethylacetophenone in 10 ml of MTBE dropwise. The mixture was stirred for two days at room temperature, and then partitioned between 0.1 N HCl and ethyl acetate (EtOAc). The EtOAc layer was washed with brine, dried over MgSO4 and concentrated to give 0.87 g of light yellow solid.

The solid obtained was dissolved in 20 of methanol, and 0.69 g of 4-hydrazinosulfonamide hydrochloride was added. The mixture was boiled on a hot plate for one hour, cooled, and the methanol removed on an aspirator. The residue was dissolved in EtOAc, washed with saturated sodium bicarbonate solution, water, and then brine, dried over MgSO4 and concentrated to give 0.73 g of light colored solid. Flash chromatography in 1:1 hexane/EtOAc gave 0.40 g of purified compound (1).

Compound 1 was dissolved in 10 ml of methanol and 10 ml of 1 N NaOH solution. After stirring overnight at room temperature, the solution was acidified with a 10% HCl solution and extracted with EtOAc. The EtOAc extract was washed with brine, dried over MgSO4 and concentrated to yield 0.33 g of the pyrazolecarboxylic acid (2).

We dissolved a mixture of 140 g of the pyrazolecarboxylic acid and 80 mg of methionine methyl ester hydrochloride was in 2 ml of dimethylformamide (DMF). To this solution we added 60 mg of imidazole and 205 mg of O-benzotriazoyl-1-yl-N,N,N′,N′-tetramethyl uranium hexaflourophosphate (HBTU). After stirring at room temperature over the weekend, the mixture was diluted with EtOAc, washed with water, saturated sodium bicarbonate solution and then brine, dried over MgSO4, and concentrated to give 128 mg of an oil. Flash chromatography on silica gel in 1:1 hexane/EtOAc gave 165 mg of purified product (5).

We next dissolved a solution of 65 mg of compound 5 in 3 ml of methanol and 2 ml of 10% NaOH. After stirring for one hour at room temperature, the solution was acidified with 10% HCl and extracted with EtOAc. The EtOAc extract was washed with brine, dried over MgSO4 and concentrated to give 25 mg of the methionine derivative (3) as a colorless solid.

Other pyrazole-containing inhibitors of this invention may be synthesized by a similar scheme with appropriate modifications that would be readily apparent to those of skill in the art.

### EXAMPLE 4

### Synthesis of Compound 10

We synthesized compound 10 according to the following scheme:

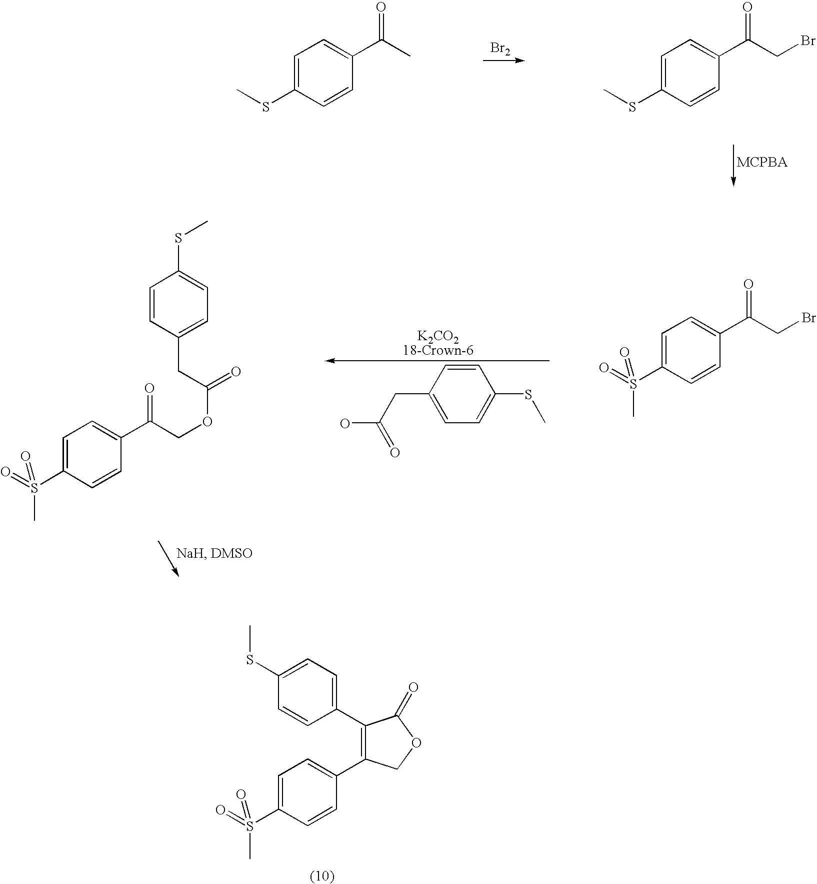

Other furan-containing inhibitors of this invention may be synthesized by a similar scheme with appropriate modifications that would be readily apparent to those of skill in the art.

### EXAMPLE 3

### Synthesis of Compounds 8 and 9

We synthesized compounds 8 and 9 according to the following scheme:

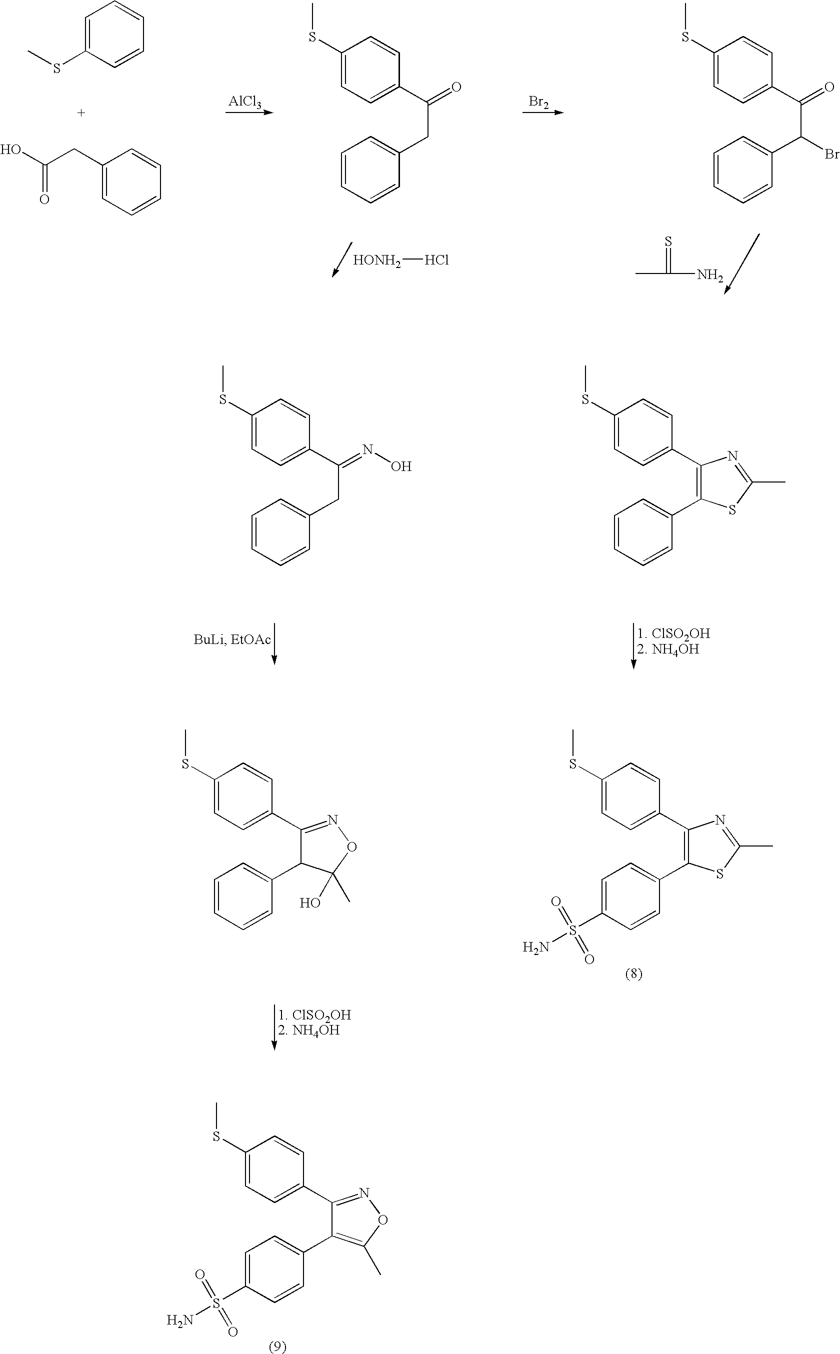

Other thiazole- and isoxazole-containing inhibitors of this invention may be synthesized by a similar scheme with appropriate modifications that would be readily apparent to those of skill in the art.

While a number of embodiments of this invention are described herein, it is apparent that my basic examples may be altered to provide other embodiments of this invention. Therefore, it will be appreciated that the scope of this invention is to be defined by the appended claims rather than by the specific embodiments which have been represented by way of example.

1. A compound of the formula; 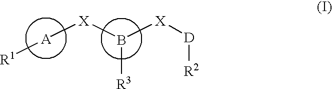
or a pharmaceutically acceptable derivative, wherein:
A is selected from aryl or heteroaryl;
ring B is selected from heterocyclyl, or carbocyclyl and is optionally benzofused;
D is selected from heterocyclyl, carbocyclyl, heteroaryl, or aryl and, when ring B is benzofused, is additionally selected from —(C1-C6alkylidene- or —(C1-C6)-heteroalkylidene-;
wherein each of A, ring B and D is optionally substituted with one or more independently selected substitutents;
each X is independently selected from a bond, —C(O)—, —CH2—, —CH═, or ═CH—;
each or R1, R2 and R3 is independently selected from hydrogen, methyl, or -L-S(O)mR4; R3 is additionally selected from CF3 or C(O)OR^{o} when B comprises only a single substitutable ring atom; and at least one of R1, R2 and R3 is selected from:
-L-S—CH3 optionally substituted with halo, O-methyl, O-ethyl, or CN;
-L-S—CH2CH3 optionally substituted with halo, O-methyl, or CN;
-L-S—CH2CH2CH3 optionally substituted with halo; or
-L-S—CH(CH3)CH3 optionally substituted with halo, wherein:
each m is independently 0, 1 or 2;
each L is independently selected from a bond, an optionally substituted (C1-C6)-alkylidene, or an optionally substituted (C1-6)-heteroalkylidene;
R^{o} is selected from hydrogen, optionally substituted C1-6 aliphatic, an unsubstituted carbocyclic ring, an unsubstituted 5-6 membered heteroaryl or heterocyclic ring, an unsubstituted aryl ring, —O-aryl, or —CH2-aryl, wherein said substituents on the aliphatic group of R^{o} are independently selected from NH2, NH(C1-4aliphatic), N(C1-4aliphatic)2, halogen, C1-4aliphatic, OH, O(C1-4aliphatic), NO2, CN, CO2H, CO2(C1-4aliphatic), O(haloC1-4 aliphatic), or haloC1-4aliphatic, wherein each of the foregoing C1-4aliphatic groups of R^{o} is unsubstituted; and
each R4 is independently selected from hydrogen, unsubstituted aryl, unsubstituted heteroaryl, unsubstituted carbocyclyl, unsubstituted heterocyclyl, —NH—(C1-C3)-aliphatic, —O—(C1-C3)-aliphatic, C(O)—(C1-C6)-aliphatic or —(C1-C6)-aliphatic or —(C1-C6)—aliphatic;

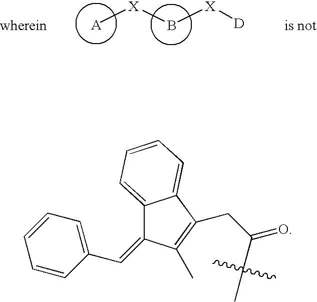

2. The compound according to claim 1, wherein A is an optionally substituted aryl ring.

3-4. (canceled)

5. The compound according to claim 1, wherein ring B is selected from an optionally substituted heterocyclyl or an optionally substituted benzofused carbocyclyl.

6. The compound according to claim 5, wherein ring B is selected from an optionally substituted five-membered heterocyclyl or an optionally substituted benzofused five-membered carbocyclyl.

7-12. (canceled)

13. The compound according to claim 1, wherein D is a ring structure and each X is a bond.

14-16. (canceled)

17. The compound according to claim 1, formula II: 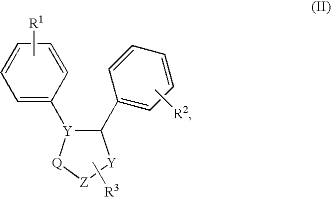
wherein:
each Y is independently selected from C or N;
Q is selected from C, N or S; and
Z is selected from C or O.

18. The compound according to claim 1, having the formula (III): 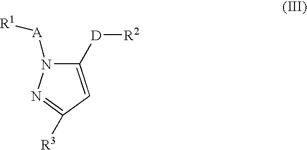
wherein:
each of A and D is aryl;
each of R1 and R2 is independently -L-S(O)mR4;
R3 is selected from:
a) —C(O)—NH—(C1-C3)-alkylidene-S-(C1-C3)-alkyl, wherein said alkylidene is optionally substituted with one or more halogens and said alkylidene is optionally substituted with —C(O)OH or —C(O)OCH3, or
b) —S(O)—NH—(C1-C3)-alkyl, wherein said alkyl is optionally substituted with one or more halogens;
at least one of R1, R2 and R3 is selected from —L—S—CH3 optionally substituted with halo, O-methyl, O-ethyl, or CN; -L—S—CH2CH3 optionally substituted with halo, Omethyl, or CN; -L—S—CH2CH2CH3 optionally substituted with halo; or -L-S—CH(CH3)CH3 optionally substituted with halo;
each L is independently selected from a bond, an optionally substituted (C1-C6)-alkylidene or, an optionally substituted (C1-C6)heteroalkylidene;
each m is independently 0, 1 or 2: and
each R4 is independently selected from hydrogen, aryl, heteroaryl, carbocyclyl, heterocyclyl, —NH—(C1-C3)-aliphatic, —NH2, —O—(C1-C3)-aliphatic, —C(O)—(C1-C6)-aliphatic or —(C1-C6)-aliphatic.

19. (canceled)

20. The compound according to claim 1, selected from: 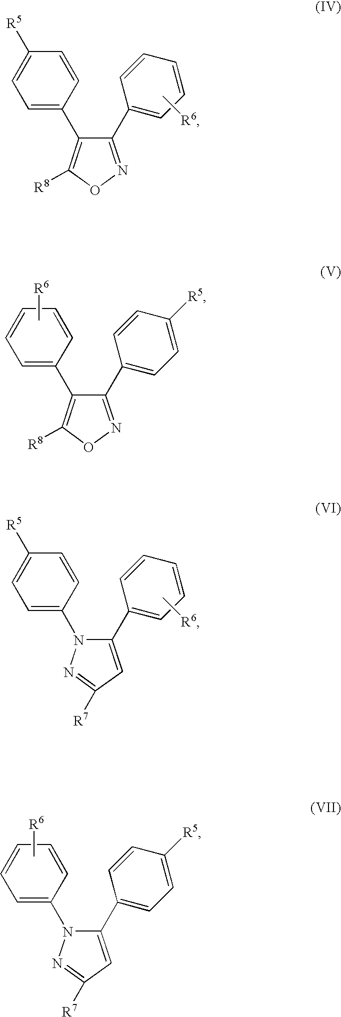 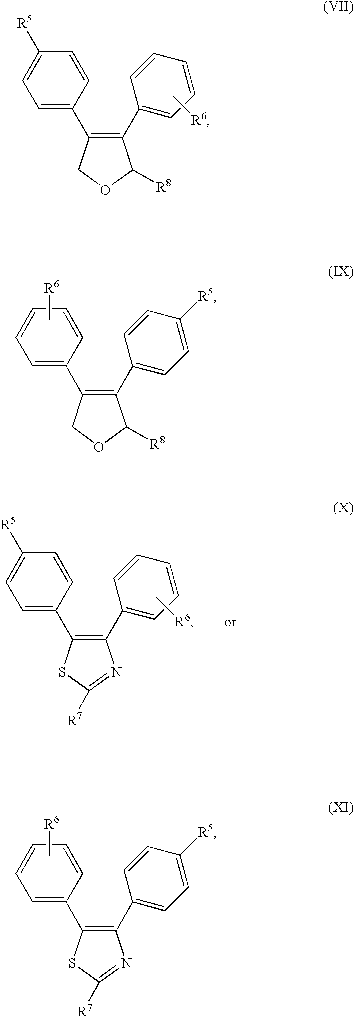
wherein:
R5 is selected from —S(O)2NH2 or —S(O)2CH3;
R6 is selected from —S—CH3 optionally substituted with halo, O-methyl, O-ethyl, or CN; —S—CH2CH3 optionally substituted with halo, O-methyl, or CN; —S—CH2CH2CH3 optionally substituted with halo; or —S—CH(CH3)CH3 optionally substituted with halo;
R7 is selected from hydrogen, -L-S(O)mR4, —C(O)OR^{o}, —CF3 or —CH3; and
R8 is selected from hydrogen, halo, ═O, —CH3 or —CN.

21. (canceled)

22. The compound according to claim 20, having the formula (VIa): 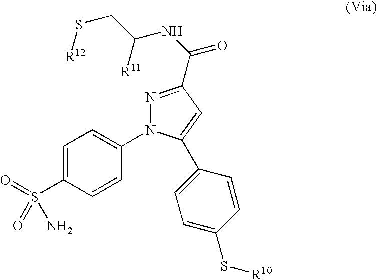
wherein:
each of R10 and R12 is selected from hydrogen, aryl, heteroaryl, carbocyclyl, heterocyclyl, —(C1-C3)-aliphatic-NH2, —(C1-C3)-aliphatic-OH, or —(C1-C6)-aliphatic; and
R11 is selected from hydrogen, —COOH, —COOCH3, —COONH2, —(C1-C6)-aliphatic or —(C1-C3)-haloaliphatic,
wherein at least one of R10 and R12 is selected from —CH3 optionally substituted with halo, O-methyl, O-ethyl, or CN; —CH2CH3 optionally substituted with halo, O-methyl, or CN; —CH2CH2CH3 optionally substituted with halo; or —CH(CH3)CH3 optionally substituted with halo.

23. The compound according to claim 20 having the formula VIb: 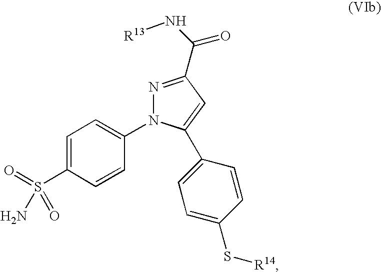
wherein:
R13 is selected from (C1-C6)-aliphatic, an unsubstituted carbocyclic ring, an unsubstituted 5-6 membered heteroaryl or heterocyclic ring, an unsubstituted aryl ring, or -L-S(O)mR4; and
R14 is selected from —CH3 optionally substituted with halo, O-methyl, O-ethyl, or CN; —CH2CH3 optionally substituted with halo, O-methyl, or CN; —CH2CH2CH2CH3 optionally substituted with halo; or —CH(CH3)CH3 optionally substituted with halo.

24. A compound selected from:

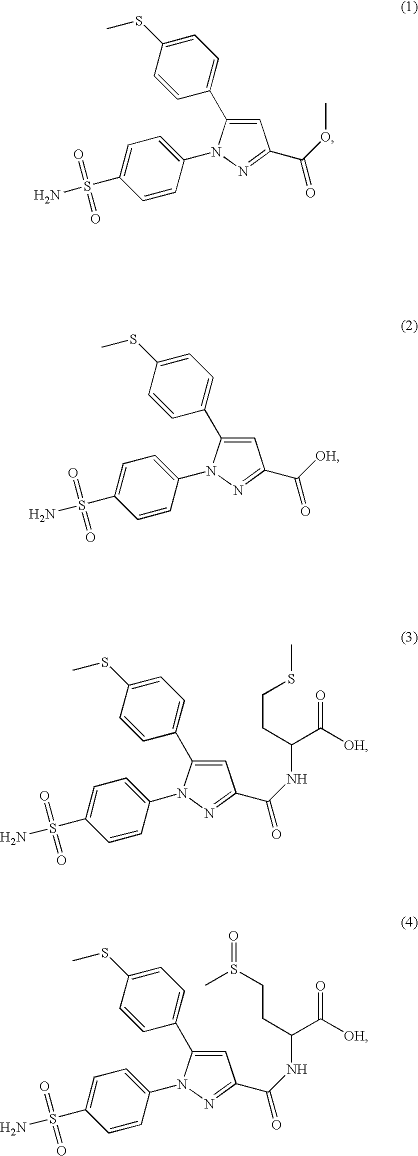

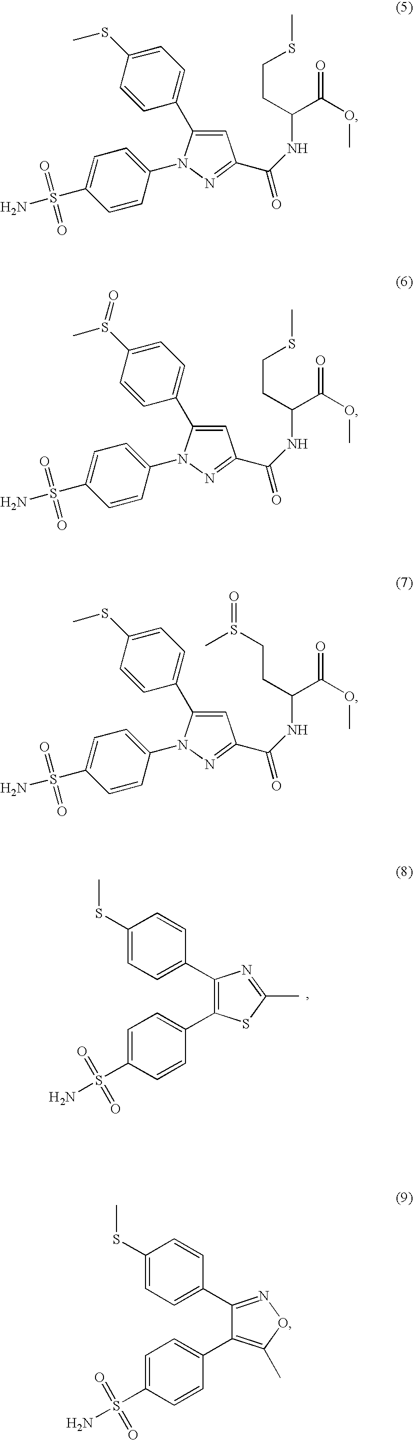

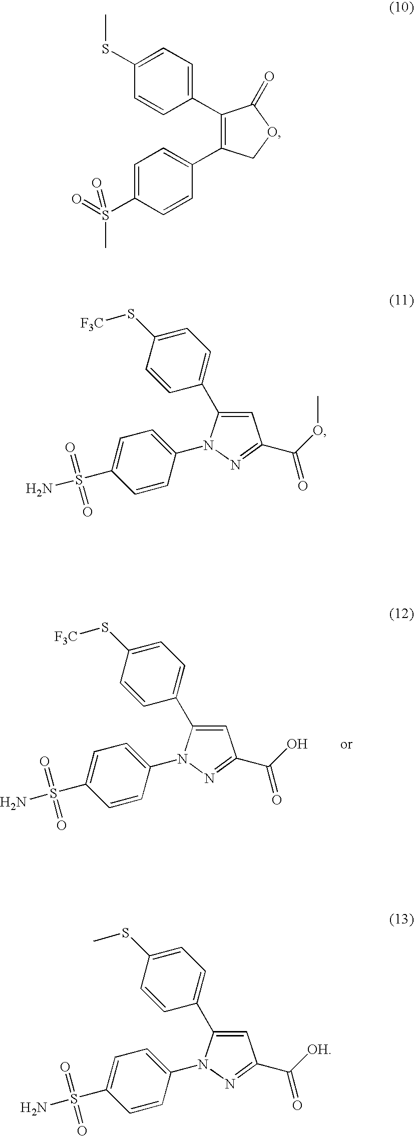

25. A composition comprising:
a) a compound according to claim 1 in an amount effective to reduce oxidative stress; and
b) a pharmaceutically acceptable carrier, adjuvant or vehicle.

26. The composition according to claim 25, additionally comprising an agent useful in a monotherapy to treat a disease or condition associated with oxidative stress in a mammal.

27-28. (canceled)

29. A method of treating or ameliorating a disease or condition caused or exacerbated by oxidative stress in a mammal comprising the step of administering to said mammal a composition according to claim 26.

30. The method according to claim 29, wherein said disease or condition is selected from arterial disease, heart disease, pulmonary disease, rheumatoid disease, eye disease, gum disease, respiratory disease, sickle cell anemia, ischemia, reperfusion injury, neurodegenerative disease, chronic inflammation, acute inflammation, cancer, reproductive dysfunction, or acute, chronic, neuropathic or inflammatory pain.

31. A method treating or ameliorating a disease or condition caused or exacerbated by both oxidative stress and inflammation in a mammal, comprising the step of administering to said mammal composition according to claim 26, wherein the compound in said composition possesses both msr-regeneratable antioxidant activity and COX-2 inhibitory activity.

32. The method according to claim 31, wherein the compound in said composition is selected from 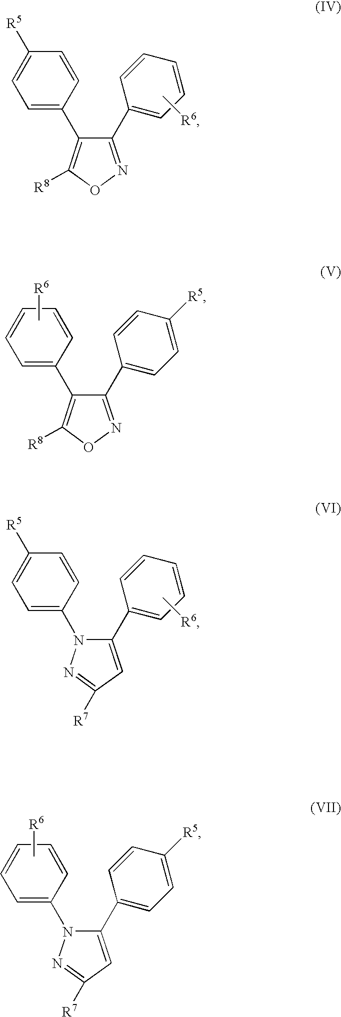 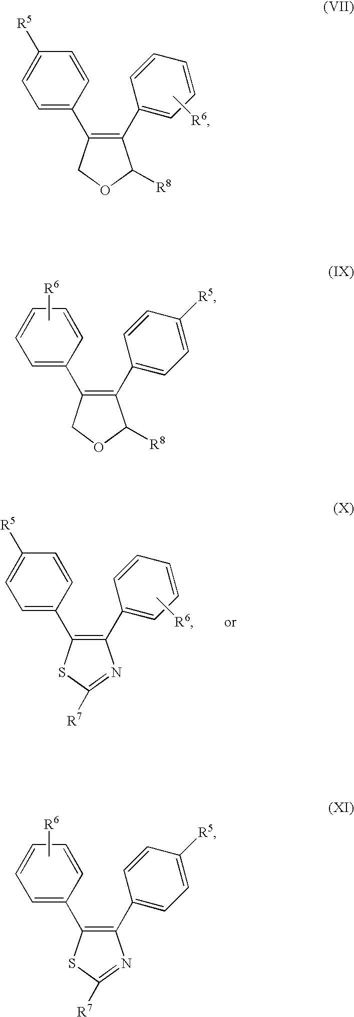
wherein:
R5 is selected from —S(O)2NH2 or —S(O)2CH3;
R6 is selected from —S—CH3 optionally substituted with halo, O-methyl, O-ethyl, or CN; —S—CH2CH3 optionally substituted with halo, O-methyl, or CN; —S—CH2CH2CH3 optionally substituted with halo; or —S—CH(CH3)CH3 optionally substituted with halo,
R7 is selected from hydrogen, -L-S(O)mR4, —C(O)OR^{o}, —CF3 or —CH3; and
R8 is selected from hydrogen, halo, ═O, —CH3 or —CN.

33. The method according to claim 31, wherein the compound in said composition is selected from

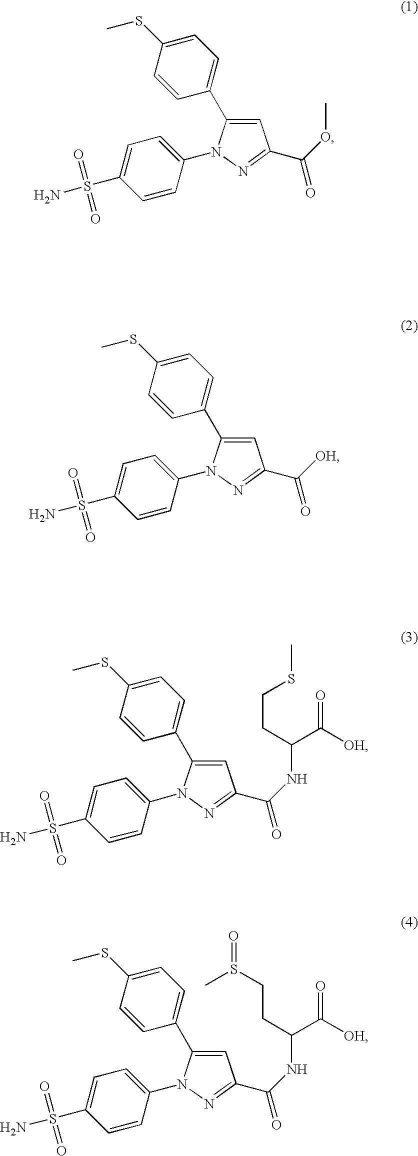

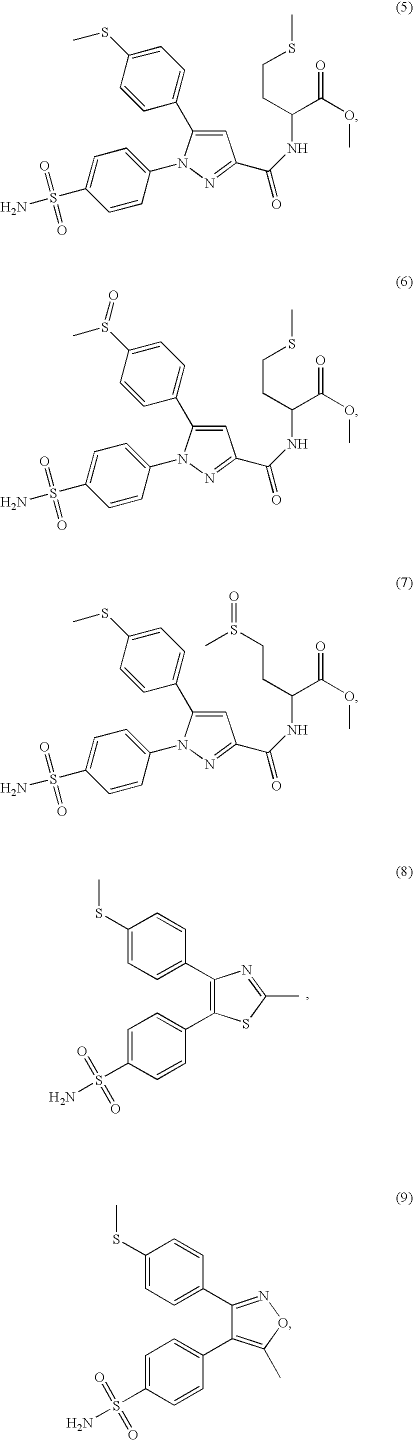

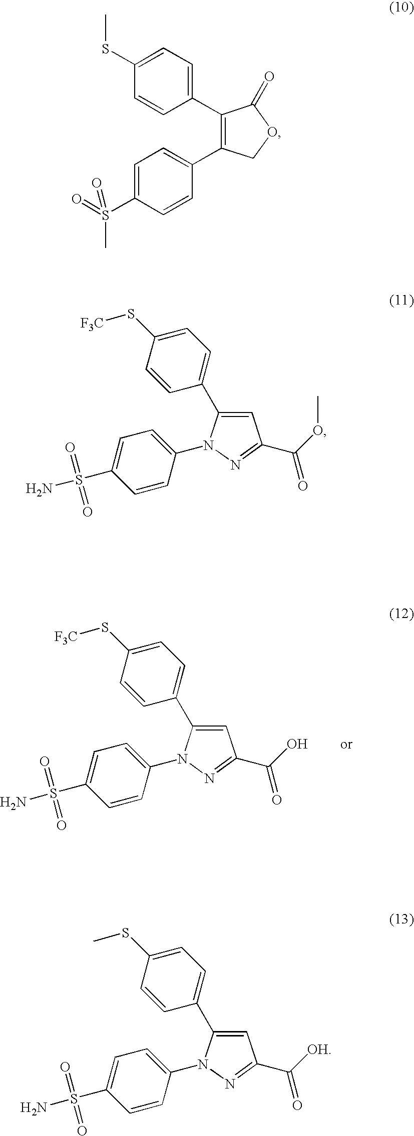

34. The method according to claim 31, wherein said disease or condition is selected from neurodegenerative disease, myocardial infarction, stroke, ischemia, acute, chronic, neuropathic or inflammatory pain.

35. The method according to claim 29, comprising the additional step of administering to said mammal another agent effective to treat said disease or condition in a monotherapy, wherein said additional agent is administered to said mammal either separately as part of a multiple dosage regimen, or as part of said composition.

36-40. (canceled)
